(19) United States
(12) Patent Application Publication
(10) Pub. No.: US 2014/0161876 A1
(43) Pub. Date: Jun. 12, 2014

(54) LIPOSOME-CONTAINING PREPARATION UTILIZING DISSOLUTION AID, AND METHOD FOR PRODUCING SAME

(71) Applicant: Takeshi Isoda, Sayama-shi (JP)

(72) Inventor: Takeshi Isoda, Sayama-shi (JP)

(73) Assignee: KONICA MINOLTA, INC., Tokyo (JP)

(21) Appl. No.: 14/232,743

(22) PCT Filed: Dec. 22, 2011
(86) PCT No.: PCT/JP2011/079852

§ 371 Date: Jan. 14, 2014

(30) Foreign Application Priority Data
Jul. 15, 2011 (JP) 2011-156769

(51) Int. Cl. A61K 9/127; C12N 15/113; A61K 31/519; A61K 31/7068

(52) U.S. Cl. CPC A61K 9/127; A61K 9/1277; A61K 31/7068; C12N 15/113; A61K 31/519
USPC 424/450; 514/49; 514/44 A; 514/249

(57) ABSTRACT

The present invention may include a production process for a preparation containing univesicular liposomes that encapsulate a highly water-soluble drug (d) having a water solubility of higher than 10 mg/mL and have a volume-average particle diameter of 50 to 200 nm utilizing a two-step emulsification method, to enhance an encapsulation ratio of the highly water-soluble drug in the liposomes produced, as compared with the conventional one. In a primary emulsification step of the two-step emulsification method, a W1/O emulsion may be prepared by the use of an aqueous phase liquid (W1) in which the highly water-soluble drug (d) and a solubilizing aid (s) having log D of not more than −1 at pH 7.4 are dissolved in an aqueous solvent (w1).

### CROSS REFERENCE TO RELATED APPLICATIONS

The present U.S. patent application is a U.S. National Phase Application under 35 U.S.C. §371 of International Application PCT/JP2011/079852 filed on Dec. 22, 2011. This application claims a priority under the Paris Convention of Japanese patent application No. 2011-156769 filed on Jul. 15, 2011, the entirety of which is incorporated herein by reference.

### TECHNICAL FIELD

The present invention relates to a liposome-containing preparation mainly used as a medicine and a production process for the same. More particularly, the present invention relates to a liposome-containing preparation and a production process for the same each of which is characterized in that a specific substance is dissolved in an inner aqueous phase of liposome.

### BACKGROUND ART

In technical fields of biology, medicines, foods, cosmetics, paints, etc., composite type fine particles called microcapsules or fine particles have been widely utilized. When a lipid is used as an emulsifying agent for the production of the composite type fine particles, the composite type fine particles are called lipid composite type fine particles. The composite type fine particles including the lipid composite type fine particles are classified into double emulsions and vesicles based on the membrane thickness thereof.

As the double emulsion of them, there is an emulsion wherein, in small oil droplets uniformly scattered in water, smaller water droplets are uniformly scattered, that is, a W/O/W emulsion (Water-in-Oil-in-Water) wherein oil droplets encapsulating water droplets are dispersed in water. The feature of this emulsion is that the membrane thickness is large because an oil phase is present between a monomolecular membrane and a monomolecular membrane. For the production of the double emulsion, a classical mechanical emulsification method or a “two-step emulsification method” utilizing an SPG (Shirasu Porous Glass) membrane emulsification method is generally used, and recently, a process for preparing W/O/W or O/W/O by extruding two kinds of unmixed fluids (W and O) that alternately run in a micro flow path into another fluid is described in a patent literature 1. By the way, it is known that when the O phase is an oil of a high boiling point, such as olive oil or decane, preparation of W/O/W easily proceeds, and the O phase shown in the working examples of the above patent literature is decane or hexadecane. On the other hand, when an organic solvent having a lower boiling point than water is used as the O phase, preparation of W/O/W is not easy, and the reason is construed to be that the power to maintain spherical shape of the particle is insufficient because of low surface tension of the organic solvent.

A liposome is a lipid composite type fine particle classified as a vesicle, and it corresponds to a structure obtained by removing the O phase from W/O/W obtained by the above production process. The vesicle is a spherical substance wherein bimolecular membranes of an amphipathic compound in parallel with each other are closed like a shell, and the feature of this substance is that the membrane thickness is small because nothing is present between a monomolecular membrane and a monomolecular membrane. Here, when an organic solvent having a lower boiling point than water is used as the O phase, it is easy to remove the solvent, and the desired liposome can be obtained. However, when an organic solvent having a higher boiling point than water is used as the O phase, it is practically difficult to remove the solvent. Production of liposomes by the “two-step emulsification method” is dilemmatic, that is, it is necessary to select an organic solvent having a low boiling point in order to remove the O phase, but in this case, there are difficulties in the production of W/O/W, and if an organic solvent of a high boiling point facilitating production of W/O/W is selected, conversion into liposomes becomes impossible, and this is a problem that is difficult to solve.

A liposome is a closed vesicle composed of a single-layer or plural-layer lipid bilayer membrane, and it can hold a water-soluble drug and a hydrophobic drug in the inner aqueous phase and inside the lipid bilayer membrane, respectively. Since the lipid bilayer membrane of the liposome is analogues to a biomembrane, it has high safety in vivo. Therefore, various uses thereof such as medicines for DDS (drug delivery system) have been noted, and research and development have been promoted.

In particular, DDS is required for gene therapy, and RNA interference (Ribonucleic Acid Interference) has been greatly noted as an innovative technique since 2001. The RNA interference is a method to produce no harmful protein by blocking a part of RNA having undergone genetic variation, with template RNA. The RNA interference can be applied to gene therapy, and diseases can be cured on the genetic level. In order to realize gene therapy, template RNA [siRNA (Small Interfering RNA)] must be introduced into a cell, first. However, the cell has a cell membrane, and therefore, when template RNA is introduced, a barrier of a cell membrane must be surmounted. In order that the gene therapy utilizing DNA (Deoxyribonucleic Acid) or RNA (Ribonucleic Acid) may exhibit a function of gene therapy, DNA or RNA must be introduced into a cell, first, similarly to the RNA interference. In recent years, there has been spreading recognition that use of virus such as retrovirus as a vector or use of a lipid vesicle (liposome) of high safety is promising.

With regard to a technique to hold a water-soluble drug and a hydrophobic drug in the inner aqueous phase and inside the lipid bilayer membrane of a liposome, respectively, there is an example wherein holding of hydrophobic drugs was relatively easily attained and medicines were put on the market. However, it is difficult to hold water-soluble drugs, and liposomes of the aforesaid gene therapy drugs have not been completed.

As one of a production process for a preparation containing liposomes to solve the aforesaid dilemma, a process comprising preparing a W/O/W emulsion by an emulsification process of two steps, then removing the oil phase (O) by evaporation and thereby forming liposomes to prepare a liposome dispersion (called microcapsulation method or two-step emulsification method) is known (non patent literature 1). However, as an example of the drug to be encapsulated, a dye called calcein is only given, and it cannot be said that the drug generality is satisfactory.

When a liposome-containing preparation composed of a dispersion of liposomes encapsulating a water-soluble drug is developed for DDS uses, the DDS effect of the liposome-containing preparation is not obtained unless a water-soluble drug is dissolved in an inner aqueous phase of a fine particle of the W/O/W emulsion, that is, an inner aqueous phase of a liposome formed from the emulsion. Even if a liposome-containing preparation in which (most of) a water-soluble drug is dissolved in an outer aqueous phase is administered, the result is almost the same as that in the case of administration of a solution obtained by only dissolving a water-soluble drug in water. For such a reason, research and development of a production process for liposomes (or liposome-containing preparation) to enhance an encapsulation ratio of a water-soluble drug (ratio of mass of water-soluble drug encapsulated in liposomes to the total mass of water-soluble drug contained in the liposome dispersion) or an absolute amount of a water-soluble drug encapsulated in liposomes have been promoted.

For example, in a patent literature 2, it is described that when a W/O/W emulsion is prepared by a microchannel emulsification method using a W/O emulsion as a disperse phase and using a Tris-HCl buffer solution as an outer aqueous phase, a “protein water-soluble emulsifying agent (casein sodium) that does not break vesicle lipid membrane” is added to the outer aqueous phase, whereby the encapsulation ratio of the substance (calcein) encapsulated in vesicles (liposomes) can be enhanced. In this patent literature 2, however, an additive to an inner aqueous phase has not been noted at all. The main object of the patent literature 2 is to secure stability of an emulsion interface in the formation of the W/O/W emulsion to thereby inhibit breakage of the emulsion, such as coalescence or layer separation. As an example of the substance to be encapsulated, calcein is only given, and any evidence to show drug generality is not described.

In a patent literature 3, it is described with regard to “multivesicular liposomes” that as an “osmotic pressure excipient” to “control the amount of a biologically active drug encapsulated in liposomes by controlling the volume osmolarity of an aqueous solution of the drug”, “glycylglycine, glucose, sucrose, trehalose, succinate, cyclodextrin, arginine, galactose, mannose, maltose, mannitol, glycine, lysine, citrate, sorbitol, dextran, sodium chloride, phosphate, a biologically active drug” or the like is added to the aqueous solution. In the patent literature 3, it has been paid attention that “multivesicular liposomes” have character of inhibiting external release of drugs by placing the drugs in the environment where the drugs are surrounded by many membranes, and working examples aiming at development of sustained release preparations have been presented.

### CITATION LIST

### Patent Literature

Patent literature 1: Japanese Patent Laid-Open Publication No 2006-272196
Patent literature 2: Japanese Patent Laid-Open Publication No 2009-280525
Patent literature 3: Japanese Patent Laid-Open Publication No 2008-044962

### Non Patent Literature

Non patent literature 1: Ishii et al. J. Dispersion Sci. Technol. vol. 9, No. 1, 1-15, 1988

### SUMMARY OF INVENTION

### Technical Problem

With regard to the production process for liposomes utilizing a two-step emulsification method, enhancement of an encapsulation ratio of water-soluble drugs or an amount of the drugs encapsulated, control of particle diameters of liposomes to a given range, etc. have been considered to be problems so far, and various means for solving the problems have been proposed. However, in order to produce a more excellent liposome-containing preparation, there is room for improvement in those means.

An object of the present invention is, in a production process for a liposome-containing preparation containing univesicular liposomes with a given particle diameter, particularly that encapsulating a highly water-soluble drug, to enhance an encapsulation ratio of the highly water-soluble drug or an amount of the drug encapsulated, as compared with the conventional one.

### Solution to Problem

The present inventors have found that when a highly water-soluble drug is used as a drug to be encapsulated, the encapsulation ratio of the highly water-soluble drug or the amount of the drug encapsulated can be enhanced by dissolving, as a “solubilizing aid”, a specific substance among substances used as additives to injections, more specifically a substance having log D of not more than −1 at pH 7.4, together with the highly water-soluble drug in an aqueous solvent constituting an inner aqueous phase of a liposome, as compared with the case where such a solubilizing aid is not contained, and the present inventors have accomplished the present invention.

In the compounds known as solubilizing aids (dissolution auxiliaries), compounds that break liposome membranes, such as isopropanol, propylene glycol and ethyl urea, are included, and there has been made no attempt to daringly add them in a production process for liposomes using a two-step emulsification method. In researches related to the present application, a pharmaceutical preparation containing a certain solubilizing aid was used in the two-step emulsification process, and as a result, an effect that the solubilizing aid strengthens a liposome membrane instead of breaking the membrane was exhibited. As a result of closer inspection of it, the present invention has been found.

That is to say, the present invention comprises the following matters.

[1] A liposome-containing preparation which is a preparation containing univesicular liposomes that encapsulate a highly water-soluble drug (d) having a water solubility of higher than 10 mg/mL and have a volume-average particle diameter of 50 to 200 nm, wherein the highly water-soluble drug (d) and a solubilizing aid (s) having log D of not more than −1 at pH 7.4 are dissolved in an inner aqueous phase (W1) of the univesicular liposome.

[2] The liposome-containing preparation as stated in [1], wherein the drug concentration of the highly water-soluble drug (d) in the liposome-containing preparation is not less than 5 mg/mL.

[3] The liposome-containing preparation as stated in [1] or [2], wherein the weight ratio (d/f) of the highly water-soluble drug (d) to a lipid component (f) constituting liposomes is not less than 0.05.

[4] The liposome-containing preparation as stated in any one of [1] to [3], wherein the highly water-soluble drug (d) is dissolved in a supersaturation state in the inner aqueous phase (W1).

[5] A production process for a preparation containing univesicular liposomes that encapsulate a highly water-soluble drug (d) having a water solubility of higher than 10 mg/mL and have a volume-average particle diameter of 50 to 200 nm, said production process comprising the following steps (1) to (4):

(1) a primary emulsification step comprising emulsifying an oil phase liquid (O), in which a lipid component (f1) is dissolved in an organic solvent (o) that is volatile under the solvent removal conditions of the following step (3), and an aqueous phase liquid (W1), in which the highly water-soluble drug (d) and a solubilizing aid (s) having log D of not more than −1 at pH 7.4 are dissolved in an aqueous solvent (w1), to produce a W1/O emulsion,

(2) a secondary emulsification step comprising emulsifying the W1/O emulsion obtained through the step (1) and an aqueous phase liquid (W2) to produce a W1/O/W2 emulsion,

(3) a solvent removal step comprising removing the organic solvent (o) contained in the oil phase liquid (O) from the W1/O/W2 emulsion obtained through the step (2) to form liposomes, and

(4) an aqueous phase substitution step comprising removing the aqueous phase liquid (W2) from a liposome dispersion obtained through the step (3) and adding an aqueous phase liquid (W3) in a smaller amount than the amount of the aqueous phase liquid (W2) removed.

[6] The process as stated in [5], wherein the secondary emulsification in the step (2) is carried out by a stirring emulsification method satisfying the condition of the following formula (e1):

0.02385<r×n/L′<0.1431 (e1)

wherein r represents a radius [m] of a stirrer, L′ represents a particle diameter [nm] of the W1/O emulsion, and n represents a number of revolutions per minute [rpm] of the stirrer.

[7] The production process as stated in [5] or [6], wherein the liposome dispersion is concentrated in the step (4) so that the drug concentration of the highly water-soluble drug (d) in the liposome-containing preparation might become not less than 5 mg/mL.

[8] The production process as stated in any one of [5] to [7], wherein in the liposome-containing preparation obtained through the step (4), the weight ratio (d/f) of the highly water-soluble drug (d) to the lipid component (f) constituting liposomes is not less than 0.05.

[9] The production process as stated in [8], wherein the aqueous phase liquid (W1) in which the highly water-soluble drug (d) is dissolved in a supersaturation state in the aqueous solvent (w1) is used in the step (1).

[10] The production process as stated in any one of [5] to [9], wherein the aqueous phase liquid (W2) in which a water-soluble emulsifying agent (r) is dissolved is used in the step (2).

[11] The production process as stated in any one of [5] to [10], wherein all of the steps (1) to (4) are carried out at a temperature in the range of 5 to 10° C.

[12] The production process as stated in any one of [5] to [11], wherein the primary emulsification in the step (1) is carried out using pulse ultrasonic waves.

### Advantageous Effects of Invention

By dissolving a solubilizing aid (s) together with the highly water-soluble drug (d) in the aqueous solvent (w1) in the primary emulsification step of the production process for a liposome-containing preparation of the present invention, the amount of the highly water-soluble drug (d) encapsulated in liposomes is increased, and therefore, a liposome-containing preparation having a high drug concentration (e.g., 5 mg/mL) and a weight ratio (d/f, e.g., not less than 0.05) of the highly water-soluble drug (d) to the lipid component (f) constituting liposomes that could not be attained in the past can be produced. By the use of a specific solubilizing aid (s), the highly water-soluble drug (d) can be sometimes dissolved in a supersaturation state in the aqueous solvent (w1), and the above weight ratio (d/g) can be further enhanced.

Moreover, by performing stirring emulsification under the given conditions in the secondary emulsification step (2), the particle size distribution of liposomes can become a normal distribution, and by dissolving the water-soluble emulsifying agent (r) in the aqueous phase liquid (W2), the W1/O/W1 emulsion and liposomes formed are stabilized, and therefore, the encapsulation ratio (quantity) of the highly water-soluble drug (d) in liposomes can be further enhanced. When all of the steps of the production process for a liposome-containing preparation of the present invention are carried out at a given low temperature, the encapsulation ratio (quantity) can be enhanced even if the highly water-soluble drug (d) has high membrane permeability. By carrying out emulsification using pulse ultrasonic waves in the primary emulsification step (1), emulsion particles having fine particle diameters (the volume-average particle diameter can be reduced to about 50 nm) and having a narrow particle size distribution can be formed. Furthermore, generation of heat accompanying the emulsification is inhibited, and all of the steps can be easily carried out at such a low temperature as above.

### DESCRIPTION OF EMBODIMENTS

### Liposome-Containing Preparation

Liposomes contained in the liposome-containing preparation of the present invention, typically liposomes contained in the liposome-containing preparation obtained by such a production process of the present invention as described below, are liposomes in which a solubilizing aid (s) is dissolved in addition to a highly water-soluble drug (d) in an inner aqueous phase (W1), and as compared with liposomes using, as an inner aqueous phase, an aqueous solvent in which no solubilizing aid is dissolved, a larger amount of the highly water-soluble drug (d) encapsulated, that is, a higher drug concentration of the liposome-containing preparation, can be attained. The drug concentration of the liposome-containing preparation depends upon solubility of the highly water-soluble drug (d) in water, encapsulation ratio of the highly water-soluble drug (d) in liposomes at the time of completion of the solvent removal step (3), concentration of liposomes in the liposome-containing preparation (amount of liposomes based on the aqueous solvent that becomes a dispersion medium for the liposomes) at the time of completion of the aqueous phase substitution step (4), etc., and the upper limit and the lower limit are not defined indiscriminately. According to the present invention, however, a usual highly water-soluble drug (d) can be contained in the liposome-containing preparation preferably in a drug concentration of not less than 5 mg/mL.

The drug concentration of the highly water-soluble drug (d) in the liposome-containing preparation is calculated from the following formula.

Drug concentration=mass of highly water-soluble drug (d) encapsulated in liposomes/volume of liposome-containing preparation

The production process for a liposome-containing preparation of the present invention is a process to produce a preparation containing univesicular liposomes. Although this process is a production process for a preparation containing univesicular liposomes, it is not meant that any multivesicular liposome should not be present in the liposomes contained in the liposome-containing preparation obtained by the production process, and the process has only to be a production process designed for the purpose of producing a preparation containing univesicular liposomes in the main. Although there is a case where multivesicular liposomes are relatively easily formed depending upon the conditions such as a blending ratio of a lipid component (f), but even in such a case, the process of the present invention is applicable, and effects such as enhancement of an encapsulation ratio of the highly water-soluble drug or an amount of the drug encapsulated, that is, enhancement of a drug concentration of the liposome-containing preparation, can be obtained.

In the present invention, the “univesicular liposome” (ULV, the same meaning as that of mononuclear liposome) indicates a liposome structure having a single inner aqueous phase, and such liposomes have a volume-average particle diameter of nanometer order, usually about 20 to 500 nm. On the other hand, the “multivesicular liposome” (MVL) indicates a liposome structure comprising a lipid membrane surrounding plural non-concentric circular inner aqueous phases, and a “multilameller liposome” (MLV) indicates a liposome structure having plural concentric circular membranes similar to “coats of onion” and having shell-like concentric circular aqueous compartments present between said membranes. The multivesicular liposomes and the multilamellar liposomes have a volume-average particle diameter of micrometer order, usually about 0.5 to 25 μm.

The sizes of the liposomes in the liposome-containing preparation of the present invention are not specifically restricted, but it is preferable to adjust them so that the volume-average particle diameter may become 50 to 200 nm. The liposomes of such sizes are almost free from a fear of blocking a capillary and can pass through a gap formed in a blood vessel in the vicinity of cancer tissue. Therefore, such liposomes are advantageously used as pharmaceuticals by administrating them to human body, and they are easily prepared.

The volume-average particle diameter of liposomes (and emulsion obtained during the course of a production process for them) is a value measured by a dynamic light scattering method. For example, an aqueous dispersion of liposomes is diluted to 10 times with PBS (phosphate-buffered saline), and particle diameters of liposomes are measured using a dynamic light scattering nanotrack particle size analyzer (UPA-EX150, Nikkiso Co., Ltd.), whereby a particle size distribution and a volume-average particle diameter can be calculated.

Substances Used for Production of Liposome-Containing Preparation

Highly Water-Soluble Drug (d)

In the present invention, the “highly water-soluble drug” to be encapsulated in liposomes is defined as a drug having a water solubility of higher than 10 mg/mL, in other words, such a drug that the amount of water required for dissolving 1 g of the drug is less than 100 mL. Such a solubility in water (level of water solubility) corresponds to ranges defined by the Japanese Pharmacopeia as “very soluble” (volume of solvent required for dissolving 1 g or 1 mL of solute: less than 1 mL), “Freely soluble” (ditto: from 1 mL to less than 10 mL), “Soluble” (ditto: from 10 mL to less than 30 mL) and “Sparingly soluble” (ditto: from 30 mL to less than 100 mL). By the Japanese Pharmacopeia, other ranges are further defined as “Slightly soluble” (ditto: from 100 mL to less than 1000 mL), “Very slightly soluble” (ditto: from 1000 mL to less than 10000 mL) and “Practically insoluble” (ditto: 10000 mL and over), and drugs having water solubility of these ranges are not applicable to the highly water-soluble drugs in the present invention.

Here, the “drugs” are substances that should be encapsulated according to the use purpose of the “liposome-containing preparation”, and not only medicines and quasi drugs (active ingredients, pharmaceutical aids, etc.) but also various substances sometimes used in fields of cosmetics and foods are also included. Of such drugs, drugs satisfying the requirements regarding the above solubility in water can be used as the highly water-soluble drugs in the present invention.

Examples of water-soluble drugs of the drugs which can be encapsulated in a liposome-containing preparation for the medical use include substances having medicinal actions, such as contrast media (non-ionic iodine compound for X-ray contrast radiography such as iohexyl, complex for MRI contrast radiography composed of gadolinium and chelating agent, etc.), anticancer drugs (pirarubicin, vincristine, taxol, mitomycin, 5-fluorouracil, irinotecan, Estracyt, epirubicin, carboplatin, intron, Gemzar, methotrexate, cytarabine, Isovorin, tegafur, cisplatin, Topotecin, pirarubicin, nedaplatin, cyclophosphamide, melphalan, ifosfamide, Tespamin, nimustine, ranimustine, dacarbazine, enocitabine, fludarabine, pentostatin, cladribine, daunomycin, aclarubicin, amurubicin, actinomycin, taxotere, trastuzumab, rituximab, gemtuzumab, lentinan, schizophyllan, interferon, interleukin, asparaginase, fosfestrol, busulfan, bortezomib, Alimta, bevacizumab, nelarabine, cetuximab, etc.), antibacterial agents (macrolide antibiotics, ketolide antibiotics, cephalosporin antibiotics, oxacephem antibiotics, penicillin antibiotics, β-lactamase antibiotics, aminoglycoside antibiotics, tetracycline antibiotics, fosfomycin antibiotics, carbapenem antibiotics, penem antibiotics), MRSA/VRE/PRSP anti-infective agents, polyene antifungal agents, pyrimidine antifungal agents, azole antifungal agents, candin antifungal agents, new quinolone antifungal agents, antioxidants, anti-inflammatory agents, blood circulation promoters, whitening agents, skin roughening inhibitors, anti-aging agents, hair-glowing promoters, moisturizing agents, hormone agents, vitamins, nucleic acid (sense strand or antisense strand of DNA or RNA, plasmid, vector, mRNA, siRNA, miRNA, etc.), proteins (enzyme, antibody, peptide, etc.), vaccine preparations (for diseases having toxoid as antigen, such as lockjaw tetanus; for diseases having virus as antigen, such as diphtheria, Japanese encephalitis, poliomyelitis, mumps and hepatitis; DNA or RNA vaccine, etc.), and pharmaceutical aids, such as dyes/fluorescent dyes, chelating agents, stabilizers and preservatives.

From such water-soluble drugs as above, those satisfying the requirements regarding the solubility in water in the present invention are selected, and they can be used as the highly water-soluble drugs in the present invention. The solubility of typical drugs is set forth in the following table.

TABLE 1

| | | Volume of | | |
|---|---|---|---|---|
| | Descriptive | solvent | | |
| | term in the | required for | | |
| | Japanese | dissolving 1 g or | Solubility in | |
| | Pharmacopeia | 1 mL of solute | water | Example |
| Highly | Very soluble | Less than 1 mL | More than 1000 mg/mL | siRNA, |
| water- | | | | iohexol |
| soluble | Freely | From 1 mL to | From more than | Estracyt, |
| drug | soluble | less than 10 mL | 100 mg/mL to | cytarabine |
| | | | 1000 mg/mL | |
| | Soluble | From 10 mL to | From more than | epirubicin, |
| | | less than 30 mL | 33.3 mg/mL to | carboplatin, |
| | | | 100 mg/mL | Gemzar |
| | Sparingly | From 30 mL to | From more than 10 mg/mL | doxorubicin, |
| | soluble | less than 100 mL | to | Isovorin, |
| | | | 33.3 mg/mL | tegafur, |
| | | | | fluorouracil |
| Non- | Slightly | From 100 mL to | From more than | cisplatin |
| highly | soluble | less than 1000 mL | 1 mg/mL to | |
| water- | | | 10 mg/mL | |
| soluble | Very slightly | From 1000 mL to | From more than | etoposide |
| drug | soluble | less than 10000 mL | 0.1 mg/mL to | |
| | | | 1 mg/mL | |
| | Practically | 10000 mL and over | From more than | paclitaxel, |
| | insoluble | | 0.01 mg/mL to | methotrexate |
| | | | 0.1 mg/mL | |

Solubilizing Aid (s)

The solubilizing aid (dissolution auxiliary) is an additive used when an active ingredient is slightly soluble in a solvent in the production of preparations such as injections. In the present invention, such a solubilizing aid (s) is a substance which exhibits an action that it can contribute to increase in the amount of the highly water-soluble drug (d) encapsulated, i.e., drug concentration of the liposome-containing preparation, when it is dissolved together with the highly water-soluble drug (d) in an aqueous solvent (w1), and is more specifically a substance which can increase the drug concentration of the liposome-containing preparation to a range that cannot be attained in the case where the substance is not added, typically not less than 5 mg/ml, when the substance is added to the aqueous solvent (w1).

It is thought that the solubilizing aid (s) can contribute to such a working effect of the present invention as above through the actions to strengthen and stabilize the liposome membrane. Moreover, the solubilizing aid can be said to be a substance capable of contributing the working effect of the present invention also from the viewpoint that the solubilizing aid enables dissolution of the highly water-soluble drug (d) in a supersaturation state in the aqueous solvent (w1).

Such solubilizing aids (s) can be selected from substances publicly known as additives to injections, and compounds having log D (the logarithm of the distribution coefficient) of not more than −1 are preferable. Of these, compounds having log D of not more than −3 are preferable because they sometimes enable dissolution of the highly water-soluble drug (d) in a supersaturation state. Examples of the compounds having log D of not less than −1 include compounds set forth in the following table, and values of log P (the logarithm of the partition coefficient) of the compounds are also set forth in the table. These values were calculated using default settings of Marvin Sketch (Chem Axon, Ltd.). In the present specification, log D is a value at pH 7.4, unless otherwise noted.

As additives to injections, isopropanol (log D=0.25), propylene glycol (log D=−0.79), monoethanolamine (log D=−0.78), etc. are publicly known, but these substances have an action to break a lipid membrane of liposome, and therefore, they are unsuitable for use as the solubilizing aids (s) of the present invention.

TABLE 2

| Solubilizing aid (s) | logP | logD (pH = 7.4) | |
|---|---|---|---|
| L-Arginine | −1.49 | −5.87 | |
| EDTA | −1.88 | −14.84 | |
| Citric acid hydrate | | −10.12 | |
| Citric acid | −1.32 | −9.47 | |
| Water | −0.65 | −0.65 | |
| Glycine | −1.15 | −3.55 | |
| L-Glutamic acid L-lysine | | −11.81 | |
| L-Glutamic acid | −0.93 | −6.32 | |
| L-Lysine | −0.71 | −5.49 | |
| Sodium salicylate | | −2.29 | |
| Salicylic acid | | −1.52 | |
| logP of ionic species | −1.55 | | |
| logP of non-ionic species | 1.98 | | |
| Structural increment | 0.63 | | |
| Sodium | −0.77 | −0.77 | |
| Tartaric acid | −1.83 | −7.89 | |
| Sucrose (saccharose) | −4.53 | −4.53 | |
| Sorbitol | −3.73 | −3.73 | |
| Trometamol | −2.71 | −4.26 | Examples |
| Lactic acid | −0.47 | −3.74 | |
| Milk sugar (lactose) | −5.34 | −5.34 | |
| Glycerol | −1.84 | −1.84 | |
| N-(2-hydroxyethyl)lactoamide | −1.75 | −1.75 | Examples |
| Grape sugar (glucose), Mannose | −3.57 | −3.57 | |
| Maltose | −5.34 | −5.34 | |
| Mannitol | −3.73 | −3.73 | Examples |
| Meglumine | −3.40 | −5.11 | Examples |

Aqueous Phase Liquids (W1), (W2) and (W3)

The first aqueous phase liquid (W1) used in the primary emulsification step constitutes an aqueous phase of the W1/O emulsion, the second aqueous phase liquid (W2) used in the secondary emulsification step constitutes an outer aqueous phase of the W1/O/W2 emulsion, and the third aqueous phase liquid (W3) used in the aqueous phase substitution step constitutes an outer aqueous phase of a final liposome-containing preparation (liposome dispersion).

The aqueous phase liquid (W1) is prepared by dissolving the highly water-soluble drug (d) and a lipid component (f1) in water or a buffer solution obtained by adding an acid and a salt for pH control to water, similarly to that in a publicly known production process for liposomes (particularly two-step emulsification method). If necessary, other solvents compatible with water, salts/saccharides for osmotic pressure control, etc. may be further dissolved. In the present specification, water or a buffer solution obtained by removing the highly water-soluble drug (d) and the solubilizing aid (s) from the aqueous phase liquid (W1), i.e., an aqueous solution or the like in which components other than the highly water-soluble drug (d) and the solubilizing aid (s) are dissolved is sometimes referred to as an “aqueous solvent (w1)”.

The aqueous phase liquid (W2) is generally water or such a buffer solution as above, similarly to that in a publicly known production process for liposomes (particularly, two-step emulsification method). If necessary, such components as above and other functional components (e.g., water-soluble emulsifying agent (f) in the present invention) may be further dissolved. In the present specification, water or a buffer solution obtained by removing the water-soluble emulsifying agent (r) from the aqueous phase liquid (W2), i.e., an aqueous solution or the like in which components other than the water-soluble emulsifying agent (r) are dissolved is sometimes referred to as an “aqueous solvent (w2)”.

As the aqueous phase liquid (W3), an aqueous solvent having the same osmotic pressure as that of the aqueous solvent (w1) constituting the aqueous solution (W1), typically the same aqueous solvent as the aqueous solvent (w1), is preferably used from the viewpoint of stability of liposomes, etc. However, it is also possible to use an aqueous solvent that is different from the aqueous solvent (w1) within limits not detrimental to the working effect of the present invention. It is not necessary to dissolve the highly water-soluble drug (d) and the solubilizing aid (s) in the same aqueous solvent as the aqueous solvent (w1), which is used as the aqueous phase liquid (W3) in the aqueous phase substitution step (4), and other conditions such as composition as a buffer solution have only to be the same.

Oil Phase Liquid (O)

The oil phase liquid (O) used in the secondary emulsification step constitutes an oil phase of the W1/O emulsion. The oil phase liquid (O) may be one composed of only an organic solvent (o), or may be one prepared by dissolving a lipid component (f2), etc. in an organic solvent (o), when needed.

It is necessary to remove the organic solvent (o) by evaporation in the step of liposome formation, and therefore, the organic solvent must be volatile at least under the conditions of the solvent removal step (3). For example, an organic solvent that has a lower boiling point than water and can be evaporated at ordinary temperature and normal pressure (if necessary, by performing stirring) is preferable. Particularly in the present invention, all of the steps including the solvent removal step (3) in the production process for a liposome-containing preparation are preferably carried out at 5 to 10° C. when stability of liposomes (enhancement of encapsulation ratio of drug having high membrane permeability) is taken into consideration, and therefore, as the organic solvent (o) in this case, preferable is an organic solvent that is evaporated at 5 to 10° C. by using reduced pressure or performing stirring if necessary. Here, the membrane permeability is an indication of ease of passing of the drug molecules through the lipid bilayer membrane of liposome. Depending upon the molecular structure of the drug, the drug molecules easily pass through a fat-soluble aliphatic chain structural part locally present inside the lipid bilayer membrane under the influence of the fat-soluble structural site, and therefore, it is not that the highly water-soluble drug cannot pass through the aliphatic chain structural part at all. This indication can be found by, for example, allowing the liposome-containing preparation to stand still at a certain temperature and measuring the drug concentrations of the inner aqueous phase and the outer aqueous phase to examine whether the drug once encapsulated moves to the outer aqueous phase with time or not. For example, as a drug having high membrane permeability, cytarabine that is an anticancer drug can be mentioned. With regard to ease of passing of the drug through the aliphatic chain structural part, influence by the structure of the compound is also an important factor, but it is a matter of common knowledge that membrane permeability of many drugs is increased by elevation of temperature because kinetic energy of lipid molecules generally rises by virtue of elevation of temperature, and this energy stands against the hydrophobic interaction of the aliphatic chain structural parts to weaken the structural strength, whereby slight gaps are formed.

As the organic solvent (o), the same organic solvent as used in a publicly known production process for liposomes (particularly two-step emulsification method including solvent removal step) can be used, and it is preferable to use an organic solvent satisfying the aforesaid volatility conditions. For example, water-insoluble organic solvents, such as hexane (n-hexane), chloroform, cyclohexane, 1,2-dichloroethene, dichloromethane, 1,2-dimethoxyethane, 1,1,2-trichloroethene, t-butylmethyl ether, ethyl acetate, diethyl ether, ethyl formate, isopropyl acetate, methyl acetate, methyl ethyl ketone and pentane, can be used. Further, water-soluble organic solvents, such as acetonitrile, methanol, acetone, ethanol and 2-propanol, ethers other than above ethers, hydrocarbons, halongenated hydrocarbons, halogenated ethers and esters can be also used. Preferable are, for example, chloroform, cyclohexane, dichloromethane, hexane, t-butylmethyl ether, ethyl acetate, diethyl ether, ethyl formate, isopropyl acetate, methyl acetate, methyl ethyl ketone, pentane, acetonitrile, methanol, acetone, ethanol and 2-propanol. When the solvent removal step (3) is carried out at a low temperature of the above range, dichloromethane (boiling point at atmospheric pressure: 40° C.), diethyl ether (ditto: 30° C.), acetone (ditto: 56.5° C.), hexane (ditto: 69° C.), etc., which are known as low-boiling solvents, are particularly preferable.

These organic solvents may be used singly, or may be used in combination of two or more kinds. For example, an organic solvent containing hexane as a main component (not less than 50% by volume), preferably an organic solvent containing hexane in an amount of not less than 60% by volume, is desirably used as the organic solvent (o) because monodispersibility of the resulting W/O emulsion particles becomes excellent.

Lipid Components (f1) and (f2)

The lipid component (f1) dissolved in the oil phase liquid (O) used in the primary emulsification step mainly constitutes an inner membrane of a lipid bilayer of liposome, and the residue can constitute also an outer membrane. On the other hand, the lipid component (f2) that is added if necessary in the secondary emulsification step or a step other than the primary emulsification step mainly constitutes an outer membrane of liposome. The compositions of the lipid components (f1) and (f2) may be the same as or different from each other.

In the present specification, the lipid component (f1) and the lipid component (f2) that is used if necessary are sometime referred to generically as a “lipid component (f)”. When the lipid component (f2) is not added in the secondary emulsification step, the lipid component (f) constituting liposome is composed of only the lipid component (f1), and when the lipid component (f2) is added in the secondary emulsification step, the lipid component (f) constituting liposome is composed of the lipid components (f1) and (f2). In the “lipid component (f) constituting liposome”, both of the later-described crystalline lipid and non-crystalline lipid are included.

The formulation of the lipid component (f) is not specifically restricted, and can be similar to the formulation of publicly known liposomes. The lipid component (f) may be a component composed of a single lipid or may be a component (mixed lipid component) composed of plural lipids. In general, the lipid component is mainly composed of phospholipids (lecithin derived from animals and plants; phosphatidylcholine, phosphatidylserine, phosphatidylglycerol, phosphatidylinositol, phosphatidic acid or glycerophosphorlipids that are their fatty acid esters; sphingophospholipid; derivatives thereof, etc.) and sterols (cholesterol, phitosterol, ergosterol, derivatives thereof, etc.) contributing to stabilization of lipid membrane. Further, glycolipid, glycol, aliphatic amine, long-chain fatty acids (oleic acid, stearic acid, palmitic acid, etc.) and other compounds imparting various functions may be added. To the lipid component (f2), lipids to impart various functions by modifying a liposome surface (outer membrane of lipid bilayer of liposome) such as PEGylated phospholipids can be also added. It is enough just to properly adjust the blending ratio of these compounds in the lipid component according to the use purpose while taking into consideration stability of the lipid membrane and properties (e.g., behavior) of liposomes in vivo.

These lipid components are usually crystalline lipids easily obtainable (in the present invention, the crystalline lipid is sometimes referred to as a “lipid component (fc)”, and particularly when crystalline lipids corresponding to the lipid component (f1) and the lipid component (f2) are indicated, they are sometimes referred to as a “lipid component (f1c)” and a “lipid component (f2c)”, respectively). Instead of this or together with this, a non-crystalline lipid having been prepared in advance can be used in the present invention (in the present invention, the non-crystalline lipid is sometimes referred to as a “lipid component (fn)”, and particularly when non-crystalline lipids corresponding to the lipid component (f1) and the lipid component (f2) are indicated, they are sometimes referred to as a “lipid component (f1n)” and a “lipid component (f2n)”, respectively). In the non-crystalline lipid component (fn), the lipid molecules are not so strongly bonded to one another as the case of the crystalline lipid component, so that the lipid molecules are apt to be separated from the lipid in a solid state, and rearrangement of lipid molecules particularly in the aqueous phase tends to be advantageously carried out. Hence, when the non-crystalline lipid component (fn) is used, formation of a W1/O/W2 emulsion is smoothly carried out, and as a result, the encapsulation ratio of a substance to be encapsulated in the form of liposomes is also enhanced. The main cause of this is considered to be that since the arrangement rate of the lipid is increased, the desired structure is obtained rapidly, and the arrangement rate is superior to the rate of breakage of the structure during the arrangement. When the non-crystalline lipid component (fn) is contained in the outer aqueous phase (that is, when the aqueous phase liquid (W2) to which the non-crystalline lipid component (f2n) has been added is used), lipid molecules are rapidly rearranged on the interface between the aqueous phase and the oil phase in the secondary emulsification, and liposomes can be preferably formed. On the other hand, when the non-crystalline lipid component (fn) is added to the aqueous phase liquid (W1) or the oil phase liquid (O), smaller liposome particles can be obtained as compared with the case of adding a crystalline lipid component, and besides, a sharp particle size distribution is obtained, so that such addition is preferable.

As the non-crystalline lipid component (fn) for use in the present invention, a lipid component of a lamellar structure can be mentioned. Here, the “lamellar structure” is known as one of liquid crystal states indicating a substance present between a liquid and a solid, and is a layer structure in which an aqueous phase and a lipid phase are alternately repeated like water/lipid/water/lipid . . . . In an amphipathic compound such as phospholipid, an aqueous phase and a lipid phase coexist in one molecule. Therefore, those compounds form a line to take such a layer structure, whereby the structure is in a stable state. The layer structure of phospholipid can be obtained in a part of a classical liposome production process using Bangham method, and an example thereof is a lipid film layer structure. As compared with a crystalline lipid that is in a stable state because of crystal lattice formed by closest packing, the layer structure is in a state where layers are repeatedly arranged by a weak interaction, so that the feature of the layer structure is that the arrangement is easily broken by an external factor such as solvent molecule and rearrangement can be carried out.

As one example of the lipid of such a “lamellar structure”, a film lipid can be also mentioned. It is known that the film lipid is prepared by, for example, completely dissolving a crystalline lipid in chloroform, placing the resulting solution in a recovery flask (also referred to as “eggplant shaped Kolben”), slowly distilling off chloroform by an evaporator and recovering lipid membrane arranged on a wall surface of the recovery flask. Such a recovery method is known as one step of the Bangham method that is a classical liposome production process.

In the present invention, the “non-crystalline lipid component (fn)” may have a usual porous structure having no lamellar structure.

To the formulation of such a non-crystalline lipid component (fn), the same formulation as in the aforesaid lipid components (f1) and (f2) is applicable, except that a non-crystalline component is used. For example, a mixed lipid obtained by the process described in Japanese Patent Publication No. 1994-74205 can be used. In the present invention, therefore, the non-crystalline lipid component (fn) may be a component composed of a single lipid, or may be a component (mixed lipid component) composed of plural lipids.

Production Process for Liposome-Containing Preparation

The production process for a liposome-containing preparation of the present invention comprises at least (1) a primary emulsification step, (2) a secondary emulsification step, (3) a solvent removal step and (4) an aqueous phase substitution step, and may comprise other steps when needed. For carrying out these steps, publicly known apparatuses and machines and other appropriate means are used, and depending upon the selection of means in these steps, it is also possible to continuously carry out steps of the primary emulsification step to the solvent removal step.

In the highly water-soluble drugs (d), those having a fear of being decomposed by high temperatures are present, and therefore, when such a drug is encapsulated as the highly water-soluble drug (d) in liposome, all of the steps (1) to (4) and other steps that are included when needed are preferably carried out under the conditions of a lower temperature than the decomposition temperature of the drug, for example, under the conditions of a temperature range of 5 to 10° C. The temperature control in each step can be carried out by the use of a publicly known appropriate means. That is to say, a solution containing raw materials used is placed together with its container in a constant temperature bath of a low temperature, and then the resulting emulsion is placed together with its container in a constant temperature bath of a low temperature, whereby heating of a drug can be avoided. Further, it is more effective that the steps (1) to (4) are automated and carried out in a low-temperature room. In such an embodiment, it becomes possible to produce liposomes using, as the highly water-soluble drug (d), a substance that is easily affected by heat, such as protein. Particularly in the production of pharmaceutical grade products under strict production control, even slight deterioration of a drug encapsulated is regarded as a problem, so that production at a low temperature can become an effective countermeasure to prevent the deterioration.

(1) Primary Emulsification Step

The primary emulsification step is a step wherein the aqueous phase liquid (1) in which the highly water-soluble drug (d) and the solubilizing aid (s) are dissolved and the oil phase liquid (O) in which the lipid component (f1) is dissolved are emulsified to produce a W1/O emulsion. In usual, the aqueous phase liquid (W1) is prepared in advance by dissolving the highly water-soluble drug (d) and the solubilizing aid (s) in the aqueous solvent (w1), and the oil phase liquid (O) is prepared in advance by dissolving the lipid component (f1) in the organic solvent (o).

As the preparation method for the W1/O emulsion in the present invention, an emulsification method used in a publicly known liposome production process (primary emulsification step), such as an ultrasonic emulsification method, a stirring emulsification method, a membrane emulsification method, a microchannel emulsification method or a method using a high-pressure homogenizer, can be used. From the viewpoint of fine particle diameter, the ultrasonic emulsification method using ultrasonic waves oscillated from an ultrasonic emulsifier or the emulsification method using a high-pressure homogenizer is preferable. When the ultrasonic emulsifier is used, it is preferable to carry out primary emulsification by using ultrasonic waves oscillated in the form of a pulse (referred to as “pulse ultrasonic waves” hereinafter). According to this method, heat generation accompanying the primary emulsification can be inhibited, so that it becomes also possible to carry out all of the steps including the steps (1) to (4) used in the present invention at low temperatures (e.g., 5 to 10° C.). Further, the energy of ultrasonic waves is intensively propagated around the ultrasonic probe. Therefore, it is thought that if the pulse is intermittent, concentration of ultrasonic waves on one place for a long time can be prevented and the ultrasonic waves rapidly become uniform, and it is thought that this contributes to decrease in a volume-average particle diameter and narrowing of a particle size distribution. When a drug that is unstable to heat or the like is encapsulated, the microchannel emulsification method in which energy required for emulsification is small or the membrane emulsification method using an SPG membrane or the like is preferable. Further, a premix membrane emulsification method comprising preparing a W1/O emulsion having a large particle diameter in advance by stirring emulsification or the like and then passing the emulsion through a membrane of a small pore diameter to prepare a W1/O emulsion having a smaller particle diameter may be used.

In the present invention, in order to encapsulate the highly water-soluble drug (d) in liposome, the highly water-soluble drug (d) and the solubilizing aid (s) are added to the aqueous solvent (w1) used in the primary emulsification step and dissolved therein.

The concentration of the solubilizing aid (s) in the aqueous phase (W1) can be controlled in the range wherein the working effect of the present invention is exerted, according to the solubility of the solubilizing aid in water, etc., and should not be defined indiscriminately, but for example, the concentration is adjusted to 5 to 150% by weight based on the weight of the highly water-soluble drug (d).

On the other hand, from the viewpoint of production of a liposome-containing preparation having a high drug concentration, the concentration of the highly water-soluble drug (d) in the aqueous phase (W1) is preferably as high as possible according to its solubility in water.

It is also possible to dissolve the highly water-soluble drug (d) in a supersaturation state in the aqueous solvent (w1), that is, the highly water-soluble drug (d) in an amount larger than its solubility in water in the aqueous solvent (w1), by the use of an appropriate means. Such a supersaturation state is preferable because the condition of such a mass ratio (d/f) as described below is easily satisfied.

The means to dissolve the highly water-soluble drug (d) in a supersaturation state in the aqueous solvent (w1) is not specifically restricted, but as a typical means in the present invention, a method of using the aforesaid solubilizing aid (s) can be mentioned. In substances given as examples of the solubilizing aids (s), a substance having a function to allow the highly water-soluble drug (d) to be dissolved in water in an amount of not less than a usual solubility, such as D-mannitol that is used in combination with Gemzar, is included. Therefore, by the use of such a substance as the solubilizing aid (s), the amount of the highly water-soluble drug (d) encapsulated can be remarkably increased. As another means, a method of dissolving the highly water-soluble drug (d) in an amorphous state or a nanoparticle crystal form in the aqueous solvent (w1) can be mentioned. By dissolving a drug substance in a crystal form, which has been purified through a recrystallization operation, in water and freeze-drying it or by dissolving it in an organic solvent and vacuum-distilling the solvent, a drug in an amorphous state can be generally obtained. The drug in a nanoparticle crystal form can be prepared referring to, for example, Elan NanoCrystal Technology. However, since the precipitation of drug crystals from the supersaturation state easily proceeds, the time for the experimental work in the suspersaturation state is generally limited to not longer than several hours. However, with progress of researches of a mechanism of precipitation, supersaturation technique has been advanced and has been able to cope with industrialization, so that an experimental work for a long time has assumed reality. That is to say, as the mechanism of precipitation, two kinds of “bulk precipitation mechanism (BPM)” wherein precipitation occurs in a solution and “surface precipitation mechanism (SPM)” wherein precipitation occurs on a solid surface have been proposed, and by judging which the highly water-soluble drug (d) belongs to, formation of proper supersaturation has been able to be realized. Actually, there is a case where by removing causes for stimulation of crystallization, such as dust, an experimental work for a half day or more can be carried out with no problem.

Drug Weight Ratio (d/f)

The weight ratio (d/f) of the highly water-soluble drug (d) to the lipid component (f) constituting liposomes is preferably higher, that is, it is preferable to encapsulate a larger amount of the highly water-soluble drug (d) in liposomes using a smaller amount of the lipid component (f).

The drug weight ratio (d/f) of the highly water-soluble drug (d) is calculated from the following formula.

Drug weight ratio=mass of highly water-soluble drug (d) encapsulated in liposomes/mass of lipid component (f) constituting liposomes

This drug weight ratio (d/g) can be set preferably to not less than 0.05, more preferably to not less than 0.5. The upper limit of the drug weight ratio varies depending upon the particle diameter of liposomes (as the particle diameter increases, the amount of the lipid component (f) constituting liposomes becomes smaller) and the solubility of the highly water-soluble drug (d) in water or the encapsulation ratio (as the solubility or the ratio rises, the amount of the highly water-soluble drug (d) encapsulated in liposomes becomes larger), and it cannot be determined unconditionally.

In order to prepare liposomes satisfying the condition of such a drug weight ratio (d/f) as above in the aqueous phase substitution step (4), it is enough just to dissolve the highly water-soluble drug (d) and the mixed lipid component (f) in amounts satisfying the condition of the desired weight ratio (d/f) in the aqueous solvent (w1) and the organic solvent (o), respectively, in the primary emulsification step (1).

An example of calculation (in the case where the drug weight ratio is 0.5 to 5) of the necessary amounts of the highly water-soluble drug (d) and the mixed lipid component (f) is given below.

The purpose of encapsulation of the water-soluble drug is achieved by dissolving the water-soluble drug in the inner aqueous phase (W1). Therefore, in the case of a drug having high water solubility, the absolute amount of the drug encapsulated can be increased by dissolving it in the inner aqueous phase (W1) in a high concentration. On the other hand, the amount of the inner aqueous phase (W1) can be properly changed, and when particles (W1/O) of given particle diameter are intended to be prepared, the amount (number of particles) of the lipid required for them can be calculated. For example, if a W1/O emulsion of 100 nm (particle volume: 0.0005 μm³) is formed and if production is performed using 1.0 mL of the W1 phase (inner aqueous phase), 2.0×10¹⁵ W1/O particles are formed according to the calculation. On the other hand, a W1/O nanoemulsion (particle surface area: 2500 nm²) of 100 nm is constituted of 0.4×10⁵ phospholipid molecules (lecithin surface area: 0.7 nm²) according to the calculation. Therefore, the amount of the lipid necessary for the primary emulsification of 1.0 mL of the drug is 2.0×10¹⁵ particles×0.4×10⁵ particles=0.8×10²⁰ particles, that is, 0.132 mmol. Since the surface area of a lipid molecule other than that of lecithin may be regarded as about 0.7 nm², it is thought that, as the total amount of the lipids, 0.132 mmol is a minimum amount required for preparing a W1/O nanoemulsion of 100 nm. For preparing liposome, 0.264 mmol that is twice the above amount is necessary, and in terms of a molecular weight of DPPC that is typical phospholipid, 193 mg is necessary.

Then, a case where a drug is dissolved in 1.0 mL to prepare liposome of 100 nm is considered. Since cytarabine is dissolved in an amount of 0.1 to 1.0 g as described in the Japanese Pharmacopeia, the drug weight ratio is 0.1 g/0.193 g to 1.0 g/0.193 g, and since iohexyl (contrast medium) is dissolved in an amount of not less than 1.0 g, the drug weight ratio is 1.0 g/0.193 g or more. This means that the amount of the lipid can be reduced to thereby effectively encapsulate the drug. This method is clinically significant from the viewpoint that the dosage of a lipid can be reduced, and by this method, a drug weight ratio of 0.5 to 5 can be accomplished. If a larger amount of the drug is dissolved, a saturation state is generally approached, and the viscosity rises. By virtue of this method, encapsulation up to 10 mPa·s in terms of a viscosity of the inner aqueous phase becomes possible.

When a particle having a size of larger than 100 nm is produced, the necessary amount of the lipid has only to be smaller than that, so that such a case is more effective.

It is also possible to encapsulate, in addition to the highly water-soluble drug (d), an oil-soluble drug in a lipid membrane of liposome, when needed. In this case, it is enough just to dissolve the oil-soluble drug in the organic solvent (o) in the primary emulsification step.

pH of the aqueous solvent (w1) is usually adjusted to a range of 3 to 10, and for example, when oleic acid is used for the mixed lipid component, pH of the aqueous solvent is preferably 6 to 8.5. In order to adjust pH, it is enough just to use an appropriate buffer solution.

Other conditions in the primary emulsification step, such as a mass ratio of the mixed lipid component (f1) to the organic solvent (o), a volume ratio between the organic solvent (o) and the aqueous solvent (w1) and a volume-average particle diameter of the W1/O emulsion, can be properly controlled in accordance with a publicly known production process for liposomes (primary emulsification step) while taking into consideration the conditions of the subsequent secondary emulsification step, the form of liposome finally prepared, etc. In usual, the mass ratio of the mixed lipid component (f1) to the organic solvent (o) is 1 to 50% by mass, and the volume ratio between the organic solvent (o) and the aqueous solvent (w1) is 100:1 to 1:2, but they can be properly controlled while taking into consideration the aforesaid condition of the mass ratio of the highly water-soluble drug (d) to the lipid component (f) constituting liposomes. The volume-average particle diameter of the W1/O emulsion is preferably 50 to 1,000 nm, more preferably 50 to 200 nm.

(2) Secondary Emulsification Step

The secondary emulsification step is a step wherein the W1/O emulsion obtained through the above-mentioned primary emulsification step and an aqueous phase liquid (W2) are emulsified to prepare a W1/O/W2 emulsion.

Of the mixed lipid component (f1) having been added in the primary emulsification, the residue that has not been orientated on the W/O interface, or a mixed lipid component (f2) that is added in the secondary emulsification when needed is orientated on the O/W interface, whereby a W1/O/W2 emulsion is formed.

The mixed lipid component (f2) that is used when needed may be added to any one of the aqueous phase liquid (W2) and the W1/O emulsion. For example, when the mixed lipid component (f2) is mainly composed of a water-soluble lipid, it is possible that the water-soluble lipid is dissolved in an aqueous solvent (w2) to prepare an aqueous phase liquid (W2) in advance and the W1/O emulsion is added to the aqueous phase liquid to perform an emulsification treatment. It is also possible that after preparation of the W1/O/W2 emulsion or after the later-described solvent removal step (3), the mixed lipid component (f2) is added. On the other hand, when the mixed lipid component (f2) is mainly composed of an oil-soluble lipid, it is possible that the oil-soluble lipid is added to an oil phase liquid (O) of the W1/O emulsion and dissolved therein in advance, and the resulting solution and an aqueous phase liquid (W2) are subjected to an emulsification treatment.

The W1/O/W2 emulsion can be prepared also by emulsifying the W1/O emulsion obtained through the aforesaid step (1) and the aqueous phase liquid (W2) to which a non-crystalline mixed lipid component (f2n) has been added. In this case, there is an advantage that the encapsulation ratio of the highly water-soluble drug encapsulated in liposome is enhanced as compared with the case of adding a crystalline lipid component (f2c) because the non-crystalline mixed lipid component (f2n) has been added to the aqueous phase liquid (W2),

Here, the non-crystalline lipid component (fn) can be added not only to the aqueous phase liquid (W2) but also to the W1/O emulsion. In this case, the non-crystalline mixed lipid component (fn) takes a dissolved or dispersed state in the W1/O emulsion.

The method for preparing the W1/O/W2 emulsion is not specifically restricted, and a conventional method for preparing a W1/O/W1 emulsion can be adopted. The conditions in the secondary emulsification step other than the below-described matters, such as a volume ratio between the W1/O emulsion and the aqueous solvent (w2) and a volume-average particle diameter of the W1/O/W2 emulsion, can be properly controlled in accordance with a publicly known production process for liposomes (secondary emulsification step) while taking the use purpose of the finally prepared liposomes, etc. into consideration

For example, in order to inhibit breakage of droplets during the emulsification operation and leakage of the encapsulated substance from the droplets, it is preferable to use a microchannel emulsification method in which high mechanical shear force is not necessary for the emulsification treatment. In the microchannel emulsification method, a microchannel emulsification apparatus module constituted of a silicon microchannel substrate and a glass plate placed over the top of the substrate is used. An exit side part of a groove type microchannel constituted of the substrate and the glass plate or an exit side part of a straight-through type microchannel manufactured on the substrate is filled with the outer aqueous phase (W2), and the W1/O emulsion is forced into the microchannel at the entrance side of the microchannel, whereby a W1/O/W2 emulsion is formed. As the substrate, any of various types such as dead end type, cross flow type and straight-through type can be used.

Further, a membrane emulsification method in which the W1/O emulsion is passed through an emulsification membrane and dispersed in the form of droplets into the outer aqueous phase (W2) to prepare a W1/O/W2 emulsion can be also used. In particular, a membrane emulsification method using an emulsification membrane formed from SPG (Shirasu Porous Glass) having fine pores with a diameter of about 0.1 to 5.0 μm is preferable, and this method can be an industrially advantageous method because the cost is low and the throughput is large.

After the W1/O/W2 emulsion is obtained through the membrane emulsification using the above method or another method, membrane treatment of the W1/O/W2 emulsion may be carried out once or plural times using a membrane that is the same as or different from the membrane used in the membrane emulsification, in order to enhance monodispersibility of the average particle diameter of the W1/O/W2 emulsion. Particularly when membrane treatment is carried out using a membrane having a smaller pore diameter than that of the membrane used in the membrane emulsification, burden on the membranes (pressure necessary for passing of emulsion through the membranes) of the membrane emulsification and the membrane treatment can be reduced as compared with the case where the W1/O/W2 emulsion having desired volume-average particle diameter and monodispersibility is prepared by membrane emulsification of one time without performing membrane treatment. By virtue of this, lengthening of the membrane life and shortening of the treatment time required for the secondary emulsification step can be achieved, so that this is advantageous in enhancement of productivity of liposomes and reduction of cost.

Stirring Emulsification Method

In the secondary emulsification step (2) of the production process for a liposome-containing preparation of the present invention, a W1/O/W2 emulsion capable of providing liposomes of a sharp particle size distribution can be prepared even by the use of stirring emulsification having a possibility of occurrence of mechanical shear force.

For the stirring emulsification, methods and apparatuses used for mixing fluids of two or more liquids can be used. For example, as stirring apparatuses, those of various forms are present. There are many apparatuses to simply rotate a stirrer in the form of a bar, a plate or a propeller at a constant rate in one direction in a tank, but apparatuses to intermittently rotate or reversely rotate a stirrer are present. Under special circumstances, there are made various devices such that plural stirrers are arranged in parallel and alternately subjected to reverse rotation and that a protrusion or a plate combined with a stirrer is fitted to the tank side to increase shear stress generated by the stirrer. There are various means to transmit power to a stirrer, and most of them are means to rotate a stirrer through a rotating shaft, but a magnetic stirrer to transmit power by rotating a stirrer that encloses a magnet therein and is coated with Teflon (trademark), from the outside of the container by means of a magnetic field is also present.

When the W1/O emulsion obtained through the primary emulsification step (1) and the aqueous solvent (w2) are mixed and emulsified, it is preferable to carry out stirring emulsification satisfying the condition of the following formula (e1) in the present invention.

0.02385<r×n<L′<0.1431 (e1)

In the formula (e1), r represents a radius [m] of a stirrer, L′ represents a particle diameter [nm] of the W1/O emulsion, and n represents a number of revolutions per minute [rpm] of the stirrer.

Here, the formula (e1) was designed on the basis of the following formula

τ (shear force)=μ (viscosity)×ν (velocity)/L (length) (e2)

that is one of Newton's law indicating momentum accompanying transfer of a fluid and on the basis of some hypotheses described below, and certainty was verified by experiments.

As previously described, the mixing emulsification in the secondary emulsification step (2) is promoted also by the shear phenomenon due to stirring, and it is promoted also by a tearing phenomenon in a microchannel. This tearing phenomenon is taken as a phenomenon brought about by a force called surface tension of a fluid, and the magnitude of this force is measured by, for example, Sugiura, Langmuir 2001, 5562. That is to say, the measured surface tension in the formation of olive oil droplets in a microchannel was 4.5 mN/m. By the way, various developments of basic equations (Euler's equations) of the hydrodynamics have been promoted by researchers, and approximations of forces acting on fluids have been presented. As the forces acting on fluids, inertial force, gravity, viscosity, interfacial tension, etc. are known, and the surface tension is approximated by the following formula.

Surface tension=interfacial tension(surface tension per unit length)×typical length of system=ρ×L

Since the droplet formed has a diameter of 17.8 μm, the interfacial tension in the system of Sugiura, et al. is calculated to be 2.5×10² [Pa].

4.5×10⁻³ [N/m]/17.8×10⁻⁶ [m]=2.5×10² [Pa]

It is unreasonable that the emulsification conditions of microchannel in which interfacial tension is dominant as the force acting on a fluid and the emulsification conditions of stirring in which shear force is dominant are treated equally, and it is difficult to mathematically verify the following hypothesis. However, validity of the formula (e1) has been finally confirmed by the experimental verifications. The hypothesis is an assumption that by giving force equivalent to the interfacial tension as shear force in the stirring, a similar tearing phenomenon takes place. That is to say, when the shear force is assumed to be τ=2.5×10² [Pa], the intermediate value of viscosity μ between water and hexane is assumed to be μ=0.005 (=0.5×10³) [PaS], and L is assumed to be 10 times the W1/O emulsion particle diameter, the above formula (e2) can be represented by the following formula using a radius r [m] of the stirrer, a particle diameter L′ [nm] of the W1/O emulsion and a number of revolutions per minute [rpm] of the stirrer.

2.5×10²=0.0005×(2π×r×n/60)/(10×L′×10⁻⁹) (e2′)

Here, L is assumed to be 10 times the W1/O emulsion particle diameter because it is presumed that by a force that shears particles having a particle diameter of about 10 times the W/O emulsion particle diameter, the W1/O emulsion is not sheared.

When the formula (e2′) is further converted, the following calculation is obtained.

r×n/L′=(2.5×10²)×(10×10⁻⁹)/(0.0005×2π)×60≈0.0478

Here, if the assumption that the shear force is equivalent to the interfacial tension is changed to an assumption that it is about 0.5 time to 3 times the interfacial tension, r×n/L′ also becomes about 0.5 time to 3 times the value of 0.0478 correspondently to this, and the following formula is derived.

0.0478×0.5<r×n/L′<0.0478×3

That is to say, the following formula is derived.

0.02385<r×n/L′<0.1431 (e1)

In the present invention, the number of revolutions per minute of the stirrer is preferably 100 to 10000 from the viewpoint of stirring operability.

In the case of fluids of low viscosity, there are apparatuses to stir the interior of a tank by pressurizing a fluid in the tank or external air by means of a pump installed outside the tank and thereby vigorously blowing it into the tank without using a stirrer, such as an aeration apparatus of a small aquarium and an industrial spray drying apparatus. As pulverizers called mills, there are hammer mill, pin mill, Ongumiru, CoBall mill, Aspec mill, ball mill, jet mill, roll mill, colloid mill, disper mill, etc., and these are apparatuses to mix a fluid by an action of mechanical force such as compression force, pressing force, expansion force, shear force, impact force or cavitation force. In the present invention, therefore, stirring may be carried out using these apparatuses instead of a stirrer. In addition to such mechanical means, electrical stirring methods can be also used.

Water-Soluble Emulsifying Agent (r)

To the aqueous phase liquid (W2) used in the secondary emulsification step, a water-soluble emulsifying agent (r) which can further contribute to enhancement of an encapsulation ratio of the highly water-soluble drug and effective formation of univesicular liposomes and does not break the liposome lipid membrane may be added in a proper amount, when needed.

Examples of typical water-soluble emulsifying agents (r) include proteins, polysaccharides, ionic surface active agents and nonionic surface active agents. Since the polysaccharides have relatively low orientation property onto the interface of the W1/O/W2 emulsion, namely, interface between the W1/O emulsion (particles) that is a primary emulsification product and the outer aqueous phase (W2), they are distributed into the whole outer aqueous phase (W2) so that the particles in the W1/O/W2 emulsion should not be joined together, whereby the liposomes are stabilized. Proteins and the nonionic surface active agents have relatively high orientation property onto the interface of the W1/O/W2 emulsion and enclose the W1/O emulsion (particles) like protective colloids, whereby the liposomes are stabilized. If the particles in the W1/O/W2 undergo coalescence and have larger particle diameters, solvent removal by a drying-in-liquid method is carried out non-uniformly, and the encapsulated drug is liable to leak, that is, the liposomes are destabilized. However, proteins can inhibit such destabilization due to such coalescence, and they contribute to enhancement of efficiency of formation of univesicular liposomes and encapsulation ratio of the drug. The nonionic surface active agents orientated on the interface of the W1/O/W2 emulsion enable loosening of individual liposomes when the liposomes are formed with removal of the solvent, and they also contribute to enhancement of efficiency of formation of univesicular liposomes and encapsulation ratio of the drug.

Examples of proteins include gelatin (soluble protein obtained by denaturing collagen by heating), albumin and trypsin. Gelatin usually has a distribution of molecular weight of several thousands to several millions, and for example, gelatin having a weight-average molecular weight of 1,000 to 100,000 is preferable. Gelatin that is on the market for medical use or foodstuffs can be used. Examples of albumins include egg albumin (molecular weight: about 45,000), serum albumin (molecular weight: about 66,000, bovine serum albumin) and lactoalbumin (molecular weight: about 14,000, α-lactoalbumin), and for example, dried desugared albumin that is egg albumin is preferable.

Examples of the polysaccharides include dextran, starch, glycogen, agarose, pectin, chitosan, carboxymethyl cellulose sodium, xanthan gum, locust bean gum, guar gum, maltotriose, amylose, pullulan, heparin and dextrin, and for example, dextran having a weight-average molecular weight of 1,000 to 100,000 is preferable.

Examples of the ionic surface active agents include sodium cholate and sodium deoxycholate.

Examples of the nonionic surface active agents include alkyl glucosides such as octyl glucoside, polyalkylene oxide-based compounds such as products of “Tween 80” (Tokyo Chemical Industry Co., Ltd., polyoxyethylene sorbitan monooleate, molecular weight: 1309.68) and“Pluronic F-68” (BASF, polyoxyethylene(160) polyoxypropylene(30) glycol, number-average molecular weight: 9600), and polyethylene glycols having a weight-average molecular weight of 1000 to 100000. As products of polyethylene glycols (PEG), “Unilube” (NOF Corporation), GL4-400NP, GL4-800NP (NOF Corporation), PEG200,000 (Wako Pure Chemical Industries, Ltd.), Macrogol (Sanyo Chemical Industries, Ltd.), etc. can be mentioned

If the molecular weight of the water-soluble emulsifying agent (r) is too low, the water-soluble emulsifying agent is liable to permeate the lipid membrane to inhibit formation of liposomes. On the other hand, if the molecular weight thereof is too high, the rate of dispersion of the W1/O/W2 emulsion in the outer aqueous phase or orientation thereof onto the interface is lowered, and this is liable to lead to coalescence of liposomes or formation of multivesicular liposomes. On that account, the weight-average molecular weight of the water-soluble emulsifying agent is preferably in the range of 1,000 to 100,000. When the weight-average molecular weight is in this range, the encapsulation ratio of the highly water-soluble drug in liposomes is good.

When the water-soluble emulsifying agent (r) is used as above, the conditions such as an amount of the water-soluble emulsifying agent added to the aqueous solvent (w2) are not specifically restricted, and it is enough just to use appropriate conditions in accordance with a publicly known production process for liposomes.

(3) Solvent Removal Step

The solvent removal step is a step wherein the organic solvent (o) contained in the oil phase (O) of the W1/O/W2 emulsion obtained through the secondary emulsification step (2) is removed to form liposomes having a lipid bilayer membrane composed of the mixed lipid component (f1) and the mixed lipid component (f2) that is added when needed. It is thought that with progress of removal of the organic solvent, hydration of the lipid constituting liposomes proceeds, and the multivesicular liposomes are loosened and take a state of univesicular liposomes, or tearing takes place from the position near the interface of the W1/O/W2 emulsion to form univesicular liposomes.

In the solvent removal step, it is preferable to use a method (drying-in-liquid method) comprising recovering the W1/O/W2 emulsion, transferring it into an open container and evaporating the organic solvent (o) contained in the W1/O/W2 emulsion to remove the organic solvent.

In the drying-in-liquid method, operations of stirring, temperature control (heating or cooling), pressure reduction, etc. may be added when needed, and in this case, an apparatus (evaporator or the like) equipped with means of stirring, temperature control, pressure reduction, etc. may be used.

The solvent removal can be carried out while allowing the W1/O/W2 emulsion to stand still in the open container, but by stirring the emulsion, solvent removal proceeds more uniformly and the gas-liquid interface is widened, whereby the time required for the solvent removal is shortened. When the W1/O/W2 emulsion is prepared by the stirring emulsification method in the secondary emulsification step, stirring can be continued thereafter to remove the solvent, that is, it is also possible to carry out the secondary emulsification step and the solvent removal step continuously.

It is enough just to control the temperature conditions within a range wherein the compound can be evaporated without bumping, according to the type of a compound used as the organic solvent (o). The temperature is preferably in the range of 0 to 60° C., more preferably 0 to 25° C., particularly preferably 5 to 10° C.

The reduced pressure conditions are preferably set within the range of the saturated vapor pressure of the organic solvent (o) to atmospheric pressure, and is more preferably set within the range of +1% to 10% of the saturated vapor pressure of the solvent. The temperature control and the pressure reduction operation may be carried out in combination so that the organic solvent (o) should not undergo bumping, and for example, when a drug that is easily affected by heat is encapsulated in liposomes, it is preferable to remove the solvent at lower temperatures under the reduced pressure conditions.

In the liposomes obtained by such a production process (two-step emulsification method) as above, multivescular liposomes derived from the W/O/W emulsion are sometimes contained in a certain proportion, and in order to decrease them, it is effective to carry out stirring, pressure reduction or a combination of them. For example, by carrying out pressure reduction and stirring for a longer time than the time required for removal of most of the solvent, hydration of the lipid constituting liposomes proceeds, and the multivesicular liposomes can be loosened and take a state of univesicular liposomes without bringing about leakage of the encapsulated substance.

(4) Aqueous Phase Substitution Step

The aqueous phase substitution step is a step wherein the aqueous phase liquid (W2) is removed from the liposome dispersion obtained through the solvent removal step (3) and an aqueous phase liquid (W3) is added to produce a liposome preparation. The main purpose of the aqueous phase substitution step is to remove the water-soluble emulsifying agent (r) that is sometimes contained in the aqueous phase liquid (W2). In the present invention, however, there is a case where the amount of the aqueous phase liquid (W3) added is made smaller than the amount of the aqueous phase liquid (W2) removed in this aqueous phase substitution step. In such a case, this aqueous phase substitution step also has a character of a concentration step practically.

Here, it does not matter what method is used to remove the aqueous phase liquid (W2) as far as the liposomes are not broken, and for example, the removal can be carried out by subjecting the liposome dispersion obtained through the step (3) to ultracentrifugation or ultrafiltration. In the case of small-quantity production, ultracentrifugation is thought to be effective, and in the case of mass production, ultrafiltration is thought to be effective.

The aqueous phase liquid (W3) is composed of an aqueous solvent (w3) which is the same as the aqueous solvent (w1) or which is different from the aqueous solvent (w1) within limits not detrimental to the working effect of the present invention, as described in the aforesaid section “Aqueous phase liquids (W1), (W2) and (W3)”. The aqueous solvent (w3) used as the aqueous phase liquid (W3) has only to be the same as the aqueous solvent (w1) in other conditions such as composition as a buffer solution, and in the aqueous phase liquid (W3), the highly water-soluble drug (d) does not need to be dissolved.

The amount of the aqueous solvent (w3) added can be controlled according to the drug concentration of the desired liposome-containing preparation. In order to increase the drug concentration, it is enough just to make the amount of the aqueous solvent (w3) added as small as possible. Practically, it is necessary to add the aqueous solvent (w3) in a minimum amount required for forming a dispersed state of fine particle liposomes containing the inner aqueous phase W1, and the amount added is thought to be equal to the amount of W1 or more. Therefore, it is thought that the drug concentration of the liposome-containing preparation obtained in this step becomes a half of the concentration of the drug in the inner aqueous phase W1 or less.

The liposome-containing preparation obtained through this aqueous phase substitution step (4) takes a form wherein the liposomes encapsulating the highly water-soluble drug (d) are dispersed in the aqueous solvent (w1). Practically, all of the highly water-soluble drug (d) is encapsulated in liposomes.

(5) Arbitrary Steps

As an arbitrary step other than the steps (1) to (4), which may be included in the production process for a liposome-containing preparation when needed, there can be mentioned, for example, a granulation step using a filter wherein the particle diameters of liposomes are adjusted to a given range (volume-average particle diameter: 50 to 200 nm) and multivesicular liposomes produced or remaining as by-products by such a production process as above can be loosened to form univesicular liposomes. The multivesicular liposome has a structure in which many water droplets each having a particle diameter of about 50 to 200 nm derived from W/O are contained inside the liposome, and therefore, by passing it through a filter of a pore diameter slightly larger than the particle diameter of W/O, the multivesicular liposome can be converted into univesicular liposome having a particle diameter of about 50 to 200 nm. It is surprising that even if such an operation of the granulation step is carried out, capture of liposomes by the filter or leakage of the encapsulated substance rarely occurs. When multivesicular liposomes remain even by carrying out such an operation, they may be captured and removed by the use of a filter for particle removal. These steps are provided after the solvent removal step (3), and they may be continuously carried out subsequently to the solvent removal step (3).

Further, various steps having been used for conventional production of liposomes, such as a separation step for removing a drug or a dispersing agent liberated in the outer aqueous phase, a filtration sterilization step that is limited to a case where the liposome particle diameter is sufficiently small and a drying powdering step for enabling production of a liposome-containing preparation by forming liposomes having shapes suitable for storage and redispersing them in an aqueous solvent when used, can be mentioned as arbitrary steps. If the drying powdering step is included, the production process for a liposome-containing preparation of the present invention is transformed into a production process for a liposome dry powder.

### EXAMPLES

### Measuring Method for Particle Size Distribution of W1/O Emulsion and Liposomes

The W1/O emulsion was diluted to 10 times with a hexane/dichloromethane mixed organic solvent (volume ratio: 1/1), and then the particle size distribution was measured using a dynamic light scattering nanotrack particle size analyzer (UPA-EX150, Nikkiso Co., Ltd.). On the other hand, the liposome dispersion was used as such, and the particle size distribution was measured using the same analyzer as above.

(Measuring Method for Encapsulation Ratio of Water-Soluble Drug)

Dispersions of liposomes containing highly water-soluble drugs (cytarabine, siRNA, levofolinate) used in the examples and a water-soluble drug (etoposide) used in the comparative examples, respectively, were each separated into liposomes (solid matter) andan outer aqueous phase (supernatant) using an ultracentrifugal apparatus. The amount (a) of each water-soluble drug encapsulated in liposomes was determined by HPLC (reverse phase column: VarianPolaris C18-A (3 μm, 2×40 mm) or the like), and a value calculated from the calculation formula a/b×100 [%] using the amount (a) and the feed amount (b) of each water-soluble drug was taken as an encapsulation ratio of each water-soluble drug.

The amount (c) of the drug dissolved in W1 of the W1/O emulsion formed after the primary emulsification or the amount (d) of the drug dissolved in W1 of the W1/O/W2 emulsion formed after the secondary emulsification was also determined by HPLC (reverse phase column: VarianPolaris C18-A (3 μm, 2×40 mm) or the like) after separation of W1 using an ultracentrifugal apparatus. A value calculated from the calculation formula c/b×100 [%] or the calculation formula d/b×100 [%] was taken as an encapsulation ratio of each water-soluble drug in the W1/O emulsion or the W1/O/W2 emulsion.

### Comparative Example 1-1

### Production of W1/O Emulsion Through Primary Emulsification Step

15 mL of hexane containing 0.3 g of egg yolk lecithin “COATSOME NC-50” (NOF Corporation) having a phosphatidylcholine content of 95%, 0.152 g of cholesterol (Chol, NOF Corporation) and 0.108 g of oleic acid (OA) was used as an oil phase liquid (O), and 5 mL of a Tris-HCl buffer solution (pH: 8, 50 mmol/L) containing cytarabine (MW: 243.22, 20 mg/mL, 80 mM) was used as an inner aqueous phase liquid (W1). In a 50 mL beaker, a mixed liquid of them was placed, and using an ultrasonic dispersing apparatus (UH-600S, SMT Co., Ltd.) in which a probe having a diameter of 20 mm had been set, the mixed liquid was irradiated with ultrasonic waves (output: 5.5) at 25° C. for 15 minutes to perform emulsification treatment. Measurement was carried out in the aforesaid manner, and as a result, the W1/O emulsion obtained in this primary emulsification step was confirmed to be a monodisperse W/O emulsion having a volume-average particle diameter of about 190 nm.

(Production of W1/O/W2 Emulsion Through Secondary Emulsification Step)

Subsequently, using, as a disperse phase, the W1/O emulsion obtained through the primary emulsification step, a W1/O/W2 emulsion was prepared by the use of an SPG membrane emulsification method. That is to say, a cylindrical SPG membrane having a diameter of 10 mm, a length of 20 mm an a pore diameter of 2.0 μm was used as an SPG membrane emulsification apparatus (manufactured by SPG Technology Co., Ltd., trade name “External Pressure Type Micro Kit”), and the apparatus exit side part was filled with, as an outer aqueous phase liquid (W2), a Tris-HCl buffer solution (pH: 8, 50 mmol/L) containing purified gelatin (Nippi, Inc., Nippi high grade gelatin type AP), and at the apparatus entrance side, the W1/O emulsion was fed to prepare a W1/O/W2 emulsion. The pressure required for membrane emulsification was about 25 kPa.

(Production of Lipsomes by Removal of Organic Solvent)

Next, the W1/O/W2 emulsion was transferred into a lidless open glass container and stirred by a stirrer at room temperature for about 20 hours to evaporate hexane. After the solvent removal, the encapsulation ratio of cytarabine was 42%.

(Concentration of Liposomes by Substitution of Outer Aqueous Phase (W2))

The resulting liposome solution was subjected to ultrafiltration, and while removing the outer aqueous phase (W2), the same Tris-HCl buffer solution (w3) (pH: 8, 50 mmol/L) as the aqueous solvent (w1) was added to exclude cytarabine contained in the outer aqueous phase (W2). Finally, a liposome-containing preparation in an amount of 10 mL that was twice the volume (5 mL) of the inner aqueous phase liquid (W1) was produced. In this preparation, liposomes encapsulating cytarabine in an amount of 42% (20 [mg/mL]×5 [mL]×0.42=42 [mg]) of the feed amount were contained. The drug concentration was 4.2 mg/mL, and cytarabine was encapsulated in a proportion of 100% in liposomes. The drug weight ratio (d/f) was (20 [mg/mL]×5 [mL]×0.42/(300+152+108) [mg]=42/560=0.075.

### Example 1-1

A liposome-containing preparation was produced in the same manner as in Comparative Example 1-1, except that 5 mL of a Tris-HCl buffer solution (pH: 8, 50 mmol/L), in which D-mannose (Log D=−3.57, equal to that of glucose) that was a solubilizing aid had been dissolved in a concentration of 10 mg/mL in addition to cytarabine, was used as the inner aqueous phase liquid (W1). After the solvent removal, the encapsulation ratio of cytarabine was 62%. In the preparation after the ultrafiltration, liposomes encapsulating cytarabine in an amount of 62% (20 [mg/mL]×5 [mL]×0.62=62 [mg]) of the feed amount were contained. The drug concentration was 6.2 mg/mL, and cytarabine was encapsulated in a proportion of 100% in liposomes. The drug weight ratio (d/f) was 62/560=0.111.

### Comparative Example 1-2

A liposome-containing preparation was produced in the same manner as in Comparative Example 1-1, except that 5 mL of a Tris-HCl buffer solution (pH: 8, 50 mmol/L) containing random sequence siRNA (MW: about 13000, 100 mg/mL, about 7.7 mM) as a highly water-soluble drug instead of cytarabine was used as the inner aqueous phase liquid (W1), and a Tris-HCl buffer solution (pH: 8, 50 mmol/L) containing Pluronic (0.1 wt %) as a water-soluble emulsifying agent instead of purified gelatin was used as the outer aqueous phase liquid (W2). After the solvent removal, the encapsulation ratio of siRNA was 40%. That is to say, in the preparation after the ultrafiltration, liposomes encapsulating siRNA in an amount of 40% (100[mg/mL]×5[mL]×0.40=200 [mg]) of the feed amount were contained. The drug concentration was 20 mg/mL, and siRNA was encapsulated in a proportion of 100% in liposomes. The drug weight ratio (d/f) was 200/560=0.357.

### Example 1-2

A liposome-containing preparation was produced in the same manner as in Example 1-1, except that 5 mL of a Tris-HCl buffer solution (pH: 8, 50 mmol/L) which contained random sequence siRNA (MW: about 13000, 100 mg/mL, about 7.7 mM) as a highly water-soluble drug instead of cytarabine and in which D-mannose that was a solubilizing aid had been dissolved in a concentration of 10 mg/mL was used as the inner aqueous phase liquid (W1), and a Tris-HCl buffer solution (pH: 8, 50 mmol/L) containing Pluronic (0.1 wt %) as a water-soluble emulsifying agent instead of purified gelatin was used as the outer aqueous phase liquid (W2). After the solvent removal, the encapsulation ratio of siRNA was 66%. That is to say, in the preparation after the ultrafiltration, liposomes encapsulating siRNA in an amount of 66% (100 [mg/mL]×5 [mL]×0.66=330 [mg]) of the feed amount were contained. The drug concentration was 33 mg/mL, and siRNA was encapsulated in a proportion of 100% in liposomes. The drug weight ratio (d/f) was 330/560=0.589.

### Comparative Example 1-3

A liposome-containing preparation was produced in the same manner as in Comparative Example 1-1, except that 5 mL of a Tris-HCl buffer solution (pH: 8, 50 mmol/L) containing levofolinate (Isovorin) (MW: 511.5, 15 mg/mL, 30 mM) as a highly water-soluble drug instead of cytarabine was used as the inner aqueous phase liquid (W1). After the solvent removal, the encapsulation ratio of levofolinate was 35%. That is to say, in the preparation after the ultrafiltration, liposomes encapsulating libofolinate in an amount of 35% (15 [mg/mL]×5 [mL]×0.35=26.25 [mg]) of the feed amount were contained. The drug concentration was 2.6 mg/mL, and levofolinate was encapsulated in a proportion of 100% in liposomes. The drug weight ratio (d/f) was 26.25/560=0.047.

### Example 1-3

A liposome-containing preparation was produced in the same manner as in Example 1-1, except that 5 mL of a Tris-HCl buffer solution (pH: 8, 50 mmol/L), which contained levofolinate (Isovorin) (MW: 511.5, 15 mg/mL, 30 mM) as a highly water-soluble drug instead of cytarabine and in which D-mannose that was a solubilizing aid had been dissolved in a concentration of 10 mg/mL, was used as the inner aqueous phase liquid (W1). After the solvent removal, the encapsulation ratio of livofolinate was 71%. That is to say, in the preparation after the ultrafiltration, liposomes encapsulating levofolinate in an amount of 71% (15[mg/mL]×5[mL]×0.71=53.25 [mg]) of the feed amount were contained. The drug concentration was 5.3 mg/mL, and levofolinate was encapsulated in a proportion of 100% in liposomes. The drug weight ratio (d/f) was 53.25/560=0.095.

TABLE 3
Results of Comparative Examples 1-1 to 1-3 and Examples 1-1 to 1-3
(SPG membrane emulsification method)

| | Highly | | | | Encapsulation | | Drug |
|---|---|---|---|---|---|---|---|
| | water-soluble | Solubility | Feed | Solubilizing | ratio after | Final | weight |
| | drug (D) | in water | amount | aid | solvent removal | concentration | ratio |
| Comp. | cytarabine | 200 mg/mL | 20 mg/mL | | 42% | 4.2 mg/mL | 0.075 |
| Ex. 1-1 | | | | | | | |
| Ex. 1-1 | | | 20 mg/mL | D-mannose | 62% | 6.2 mg/mL | 0.111 |
| | | | | (10 mg/mL) | | | |
| Comp. | siRNA | 500 mg/mL | 100 mg/mL | | 40% | 20 mg/mL | 0.357 |
| Ex. 1-2 | | | | | | | |
| Ex. 1-2 | | | 100 mg/mL | D-mannose | 66% | 33 mg/mL | 0.589 |
| | | | | (10 mg/mL) | | | |
| Comp. | levofolinate | 20 mg/mL | 15 mg/mL | | 35% | 2.6 mg/mL | 0.047 |
| Ex. 1-3 | (Isovorin) | | | | | | |
| Ex. 1-3 | | | 15 mg/mL | D-mannose | 71% | 5.3 mg/mL | 0.095 |
| | | | | (10 mg/mL) | | | |

### Comparative Example 2-1

A liposome-containing preparation was produced in the same manner as in Comparative Example 1-1, except that the production method for a W1/O/W2 emulsion in the secondary emulsification step was changed to a stirring emulsification method from the SPG emulsification method, and a Tris-HCl buffer solution (pH: 8, 50 mmol/L) containing Pluronic F68 (0.1 wt %) as a water-soluble emulsifying agent instead of purified gelatin was used as the outer aqueous phase liquid (W2), as described below.

(Production of W1/O Emulsion Through Primary Emulsification Step)

15 mL of hexane containing 0.3 g of egg yolk lecithin “COATSOME NC-50” (NOF Corporation) having a phosphatidylcholine content of 95%, 0.152 g of cholesterol (Chol, NOF Corporation) and 0.108 g of oleic acid (OA) was used as an oil phase liquid (O), and 5 mL of a trishyrochloric acid buffer solution (pH: 8, 50 mmol/L) containing cytarabine (MW: 243.22, 20 mg/m, 80 mM) was used as an inner aqueous phase liquid (W1). Ina 50 mL beaker, a mixed liquid of them was placed, and using an ultrasonic dispersing apparatus (UH-600S, SMT Co., Ltd.) in which a probe having a diameter of 20 mm had been set, the mixed liquid was irradiated with ultrasonic waves (output: 5.5) at 25° C. for 15 minutes to perform emulsification treatment. Measurement was carried out in the aforesaid manner, and as a result, the W1/O emulsion obtained in this primary emulsification step was confirmed to be a monodisperse W/O emulsion having a volume-average particle diameter of about 190 nm.

(Production of W1/O/W2 Emulsion Through Secondary Emulsification Step)

Subsequently, using, as a disperse phase, the W1/O emulsion obtained through the primary emulsification step, a W1/O/W2 emulsion was prepared by the use of a stirring emulsification method. That is to say, when a Tris-HCl buffer solution (pH: 8, 50 mmol/L) containing Pluronic F68 (0.1 wt %), which was an outer aqueous phase liquid (W2), was stirred at room temperature at 1000 rpm using a magnetic stirrer with a stirrer having a radius of 0.016 m (1.6 cm), the W1/O emulsion was fed, and they were stirred at room temperature for 15 minutes in such a ratio that the volume ratio between W1/O and W2 became 1:3, to prepare a W1/O/W2 emulsion. It was confirmed that cytarabine was contained in the particles.

(Production of Liposomes by Removal of Organic Solvent)

Next, the W1/O/W2 emulsion was transferred into a lidless open glass container and stirred by a stirrer at room temperature for about 20 hours to evaporate hexane. After the solvent removal, the encapsulation ratio of cytarabine was 42%.

(Concentration of Liposomes by Substitution of Outer Aqueous Phase (W2))

The resulting liposome solution was subjected to ultrafiltration, and while removing the outer aqueous phase (W2), the same Tris-HCl buffer solution (w3) (pH: 8, 50 mmol/L) as the aqueous solvent (w1) was added to exclude cytarabine contained in the outer aqueous phase (W2). Finally, a liposome-containing preparation in an amount of 10 mL that was twice the volume (5 mL) of the inner aqueous phase liquid (W1) was produced. In this preparation, liposomes encapsulating cytarabine in an amount of 42% (20 [mg/mL]×5 [mL]×0.42=42 [mg]) of the feed amount were contained. The drug concentration was 4.2 mg/mL, and cytarabine was encapsulated in a proportion of 100% in liposomes. The drug weight ratio (d/f) was 42 [mg]/(300+152+108) [mg]=42/560=0.075.

### Example 2-1

A liposome-containing preparation was produced in the same manner as in Comparative Example 2-1, except that 5 mL of a Tris-HCl buffer solution (pH: 8, 50 mmol/L), in which mannitol that was a solubilizing aid had been dissolved in a concentration of 10 mg/mL in addition to cytarabine, was used as the inner aqueous phase liquid (W1). After the solvent removal, the encapsulation ratio of cytarabine was 62%. In the preparation after the ultrafiltration, liposomes encapsulating cytarabine in an amount of 62% (20 [mg/mL]×5 [mL]×0.62=62 [mg]) of the feed amount were contained. The drug concentration was 6.2 mg/mL, and cytarabine was encapsulated in a proportion of 100% in liposomes. The drug weight ratio (d/f) was 62/560=0.111.

### Comparative Example 2-2

A liposome-containing preparation was produced in the same manner as in Comparative Example 2-1, except that 5 mL of a Tris-HCl buffer solution (pH: 8, 50 mmol/L) containing etoposide (0.2 mg/mL) that was not a highly water-soluble drug instead of cytarabine that was a highly water-soluble drug was used as the inner aqueous phase liquid (W1). After the solvent removal, the encapsulation ratio of etoposide was 33%. That is to say, in the preparation after the ultrafiltration, liposomes encapsulating etoposide in an amount of 33% (0.2[mg/mL]×5[mL]×0.33=0.33 [mg]) of the feed amount were contained. The drug concentration was 0.033 mg/mL, and etoposide was encapsulated in a proportion of 100% in liposomes. The drug weight ratio (d/f) was 0.33/560=0.0006.

### Comparative Example 2-3

A liposome-containing preparation was produced in the same manner as in Example 2-1, except that 5 mL of a Tris-HCl buffer solution (pH: 8, 50 mmol/L) containing etoposide (0.2 mg/mL) that was not a highly water-soluble drug instead of cytarabine that was a highly water-soluble drug was used as the inner aqueous phase liquid (W1). After the outer aqueous phase (W2) substitution, the encapsulation ratio of etoposide was 32%. That is to say, in the preparation after the ultrafiltration, liposomes encapsulating etoposide in an amount of 32% (0.2[mg/mL]×5[mL]×0.32=0.32 [mg]) of the feed amount were contained. The drug concentration was 0.032 mg/mL, and etoposide was encapsulated in a proportion of 100% in liposomes. The drug weight ratio (d/f) was 0.32/560=0.0006.

### Example 2-2

A liposome-containing preparation was produced in the same manner as in Comparative Example 2-1, except that 5 mL of a Tris-HCl buffer solution (pH: 8, 50 mmol/L), in which N-(2-hydroxyethyl)lactoamide (Log D=−1.75) that was a solubilizing aid had been dissolved in a concentration of 10 mg/mL in addition to cytarabine, was used as the inner aqueous phase liquid (W1). After the solvent removal, the encapsulation ratio of cytarabine was 59%. In the preparation after the ultrafiltration, liposomes encapsulating cytarabine in an amount of 59% (20 [mg/mL]×5 [mL]×0.59=59 [mg]) of the feed amount were contained. The drug concentration was 5.9 mg/mL, and cytarabine was encapsulated in a proportion of 100% in liposomes. The drug weight ratio (d/f) was 59/560=0.105.

### Comparative Example 2-4

A liposome-containing preparation was produced in the same manner as in Comparative Example 2-1, except that 5 mL of a Tris-HCl buffer solution (pH: 8, 50 mmol/L), in which propylene glycol (Log D=−0.79) that was a solubilizing aid having log D of larger than −1 had been dissolved in a concentration of 5 mg/mL in addition to cytarabine, was used as the inner aqueous phase liquid (W1). After the solvent removal, the encapsulation ratio of cytarabine was 22%. In the preparation after the ultrafiltration, liposomes encapsulating cytarabine in an amount of 22% (20 [mg/mL]×5 [mL]×0.22=22 [mg]) of the feed amount were contained. The drug concentration was 2.2 mg/mL, and cytarabine was encapsulated in a proportion of 100% in liposomes. The drug weight ratio (d/f) was 2.2/560=0.069.

### Example 2-3

1.0 mL of a liposome-containing preparation was produced in the same manner as in Comparative Example 2-1, except that the inner aqueous phase liquid (W1) was changed to 0.25 mL of an isotonic phosphoric acid buffer solution, which contained 40 mg of random sequence siRNA (MW: about 13000) as a highly water-soluble drug instead of cytarabine and in which D-mannose that was a solubilizing aid had been dissolved in a concentration of 10 mg/mL, the oil phase liquid (O) was changed to 1.25 mL of a mixed solution of dichloromethane and hexane (mixing ratio: 1:3) containing 37.5 mg of DPPC (dipalmitoylphosphatidylcholine, “MC-6060”, NOF Corporation) and 7.5 mg of DPPG (dipalmitoyl phosphatidylglycerol, “COATSOME MG-6060LA”, NOF Corporation) from 15 mL of hexane containing 0.3 g of egg yolk lecithin “COATSOME NC-50” (NOF Corporation) having a phosphatidylcholine content of 95%, 0.152 g of cholesterol (Chol) and 0.108 g of oleic acid (OA), and the concentration operation of liposomes by substitution of the outer aqueous phase (W2) was carried out by performing ultracentrifugation instead of ultrafiltration.

After the outer aqueous phase (W2) substitution, the encapsulation ratio of siRNA was 66%. That is to say, in the preparation after the ultracentrifugation, liposomes encapsulating siRNA in an amount of 66% (40 mg×0.66=26.4 [mg]) of the feed amount were contained. The drug concentration was 26.4 mg/mL, and siRNA was encapsulated in a proportion of 100% in liposomes. The drug weight ratio (d/f) was 26.4/45=0.587.

### Examples 2-4

In order to confirm whether all of the primary emulsification step, the secondary emulsification step, the solvent removal step and the aqueous phase substitution step could be carried out at low temperatures or not, the production process shown in the above Example 2-3 was carried out at low temperatures.

Specifically, in the primary specification step of Example 2-3, the temperature in the emulsification treatment by irradiation with ultrasonic waves at 25° C. for 15 minutes was changed to 5 to 10° C. In the secondary emulsification step, the temperature in the stirring at room temperature for 15 minutes was changed to 5 to 10° C. In the solvent removal step, the temperature in the stirring at room temperature for about 20 hours was changed to 5 to 10° C. In the removal of the aqueous phase liquid (W2), the temperature in the ultracentrifugation at room temperature was changed to 5 to 10° C. That is to say, all of the steps were carried out at 5 to 10° C.

As a result, results equivalent to or higher than the results of Example 2-3 could be obtained. That is to say, after the solvent removal, the encapsulation ratio of siRNA was 77%, and in the preparation after the ultracentrifugation, liposomes encapsulating siRNA in an amount of 77% (40 mg×0.7=30.8 [mg]) of the feed amount were contained, so that the drug concentration was 30.8 mg/mL, and siRNA was encapsulated in a proportion of 100% in liposomes. The drug weight ratio (d/f) was 30.8/45=0.684.

In this example, the progress was observed, and as a result, the encapsulation ratio calculated from the amount of the drug dissolved in W1 of the W1/O emulsion formed after the primary emulsification and the encapsulation ratio calculated from the amount of the drug dissolved in W1 of the W1/O/W2 emulsion formed after the secondary emulsification were 81% and 81%, respectively. On the other hand, the encapsulation ratios in Example 2-3 were 81% and 70%, respectively.

### Example 2-5

1.0 mL of a liposome-containing preparation was produced in the same manner as in Comparative Example 2-1, except that the inner aqueous phase liquid (W1) was changed to 0.25 mL of an isotonic phosphoric acid buffer solution, which contained cytarabine (MW: 243.22, 250 mg/mL, 1000 mM) in a supersaturation state and in which D-mannose that was a solubilizing aid had been dissolved in a concentration of 10 mg/mL, the oil phase liquid (O) was changed to 1.25 mL of a mixed solution of dichloromethane and hexane (mixing ratio: 1:3) containing 37.5 mg of DPPC (dipalmitoyl phosphatidylcholine, “MC-6060”, NOF Corporation), 11 mg of cholesterol (Chol, NOF Corporation) and 11 mg of DSPE-PEG2000 (distearoyl phosphatidylethanolamine polyethylene glycol, NOF Corporation) from 15 mL of hexane containing 0.3 g of egg yolk lecithin “COATSOME NC-50” (NOF Corporation) having a phosphatidylcholine content of 95%, 0.152 g of cholesterol (Chol) and 0.108 g of oleic acid (OA), and the concentration operation of liposomes by substitution of the outer aqueous phase (W2) was carried out by performing ultracentrifugation instead of ultrafiltration.

After the solvent removal, the encapsulation ratio of cytarabine was 51%. That is to say, in the preparation after the ultracentrifugation, liposomes encapsulating cytarabine in an amount of 51% (250[mg/mL]×0.25[mL]×0.51=31.875 [mg]) of the feed amount were contained. The drug concentration was 31.875/1.0=31.875 mg/mL, and cytarabine was encapsulated in a proportion of 100% in liposomes. The drug weight ratio (d/f) was 31.875/59.5=0.53.

### Example 2-6

Experiment was carried out in the same manner as in Example 2-3, except that 1.25 mL of the mixed solution of dichloromethane and hexane (mixing ratio: 1:3) containing 37.5 mg of DPPC (dipalmitoyl phosphatidylcholine, “MC-6060”, NOF Corporation) and 7.5 mg of DPPG (dipalmitoyl phosphatidylglycerol, “COATSOME MG-6060LA”, NOF Corporation) was changed to 1.25 mL of a mixed solution of dichloromethane and hexane (mixing ratio: 1:3) containing 25 mg of DPPC (dipalmitoyl phosphatidylcholine, “MC-6060”, NOF Corporation) and 5 mg of DPPG (dipalmitoyl phosphatidylglycerol, “COATSOME MG-6060LA”, NOF Corporation), and an isotonic PBS solution containing a porous lipid, said porous lipid having been prepared in advance so as to contain DPPC and cholesterol in amounts of 12.5 mg and 2.5 mg, respectively, and Pluronic F68 of 0.1% was used as the outer aqueous phase (W2).

As a result, results equivalent to or higher than the results of Example 2-3 could be obtained. That is to say, after the solvent removal, the encapsulation ratio of siRNA was 76%, and in the preparation after the ultracentrifugation, liposomes encapsulating siRNA in an amount of 76% (40 mg×0.76=30.8 [mg]) of the feed amount were contained, so that the drug concentration was 30.8 mg/mL, and siRNA was encapsulated in a proportion of 100% in liposomes. The drug weight ratio (d/f) was 30.8/45=0.684. In this example, the progress was observed, and as a result, the encapsulation ratio calculated from the amount of the drug dissolved in W1 of the W1/O emulsion formed after the primary emulsification and the encapsulation ratio calculated from the amount of the drug dissolved in W1 of the W1/O/W2 emulsion formed after the secondary emulsification were 79% and 79%, respectively. On the other hand, the encapsulation ratios in Example 2-3 were 81% and 70%, respectively, as previously described.

TABLE 4
Results of Comparative Examples 2-1 to 2-4 and Examples 2-1 to 2-6
(stirring emulsification method)

| | | | | | | | Encapsulation | | | |
|---|---|---|---|---|---|---|---|---|---|---|
| | Highly | | | | | | ratio | | | |
| | water- | Solubility | | | | | after | | | Drug |
| | soluble | in | | Feed | | Solubilizing | solvent | Final | | weight |
| | drug (D) | water | | amount | | aid | removal | concentration | | ratio |
| Comp. | cytarabine | 200 | mg/mL | 20 | mg/mL | | 42% | 4.2 | mg/mL | 0.075 |
| Ex. 2-1 | | | | | | | | | | |
| Comp. | etoposide | 0.5 | mg/mL | 0.2 | mg/mL | | 33% | 0.033 | mg/mL | 0.0006 |
| Ex. 2-2 | (non-highly | | | | | | | | | |
| Comp. | water-soluble | | | 0.2 | mg/mL | mannitol | 32% | 0.032 | mg/mL | 0.0006 |
| Ex. 2-3 | drug) | | | | | (10 mg/mL) | | | | |
| Comp. | cytarabine | 200 | mg/mL | 20 | mg/mL | propylene | 22% | 2.2 | mg/mL | 0.00039 |
| Ex. 2-4 | | | | | | glycol | | | | |
| | | | | | | (LogD > −0.1) | | | | |
| Ex. 2-1 | cytarabine | 200 | mg/mL | 20 | mg/mL | mannitol | 62% | 6.2 | mg/mL | 0.111 |
| | | | | | | (10 mg/mL) | | | | |
| Ex. 2-2 | cytarabine | 200 | mg/mL | 20 | mg/mL | N-(2- | 59% | 5.9 | mg/mL | 0.105 |
| | | | | | | hydroxyethyl) | | | | |
| | | | | | | lactoamide | | | | |
| | | | | | | (10 mg/mL) | | | | |
| Ex. 2-3 | siRNA | 500 | mg/mL | 160 | mg/mL | D-mannose | 66% | 26.4 | mg/mL | 0.587 |
| | | | | | | (10 mg/mL) | | | | |
| Ex. 2-4 | siRNA | 500 | mg/mL | 160 | mg/mL | D-mannose | 77% | 30.8 | mg/mL | 0.684 |
| | | | | | | (10 mg/mL) | | | | |
| Ex. 2-5 | cytarabine | 200 | mg/mL | 250 | mg/mL | D-mannose | 51% | 31.875 | mg/mL | 0.53 |
| | | | | | | (10 mg/mL) | | | | |
| Ex. 2-6 | siRNA | 500 | mg/mL | 160 | mg/mL | D-mannose | 76% | 30.8 | mg/mL | 0.684 |

(10 mg/mL)

### Reference Example A-1

A liposome-containing preparation was produced in the same manner as in Example 1-1, except that the “15 minute-ultrasonic irradiation” in the primary emulsification step was changed to “pulse ultrasonic irradiation”, the production method for the W1/O/W2 emulsion in the secondary emulsification step was changed to a “stirring emulsification method” from the “SPG emulsification method”, and the solubilizing aid was changed to mannitol from D-mannose, as described below.

(Production of W1/O Emulsion Through Primary Emulsification Step)

15 mL of hexane containing 0.3 g of egg yolk lecithin “COATSOME NC-50” (NOF Corporation) having a phosphatidylcholine content of 95%, 0.152 g of cholesterol (Chol) and 0.108 g of oleic acid (OA) was used as an oil phase liquid (O), and 5 mL of a Tris-HCl buffer solution (pH: 8, 50 mmol/L), which contained cytarabine (MW: 243.22, 20 mg/L, 80 mM) and in which mannitol that was a solubilyzing aid had been dissolved in a concentration of 10 mg/mL, was used as an inner aqueous phase liquid (W1). In a 50 mL beaker, a mixed liquid of them was placed, and using an ultrasonic dispersing apparatus (UH-600S, SMT Co., Ltd., output: 5.5) in which a probe having a diameter of 20 mm had been set, the mixed liquid was subjected to pulse ultrasonic irradiation wherein irradiation for 1 minute and non-irradiation for 1 minute were alternately repeated, at 25° C. to perform emulsification treatment. Measurement was carried out in the aforesaid manner, and as a result, the W1/O emulsion obtained in this primary emulsification step was confirmed to be a monodisperse W/O emulsion having a volume-average particle diameter of 50 nm.

(Production of W1/O/W2 Emulsion Through Secondary Emulsification Step)

Subsequently, using, as a disperse phase, the W1/O emulsion obtained through the primary emulsification step, a W1/O/W2 emulsion was prepared by the use of a stirring emulsification method. That is to say, when a Tris-HCl buffer solution (pH: 8, 50 mmol/L) containing purified gelatin (Nippi, Inc., Nippi high grade gelatin type AP) was stirred at 50 rpm using a magnetic stirrer with a stirring blade having a radius of 0.03 m (3 cm), the W1/O emulsion was fed, and they were stirred in such a ratio that the volume ratio between W1/O and W2 became 1:3, to prepare a W1/O/W2 emulsion. It was confirmed that cytarabine was contained in the particles.

(Production of Lipsomes by Removal of Organic Solvent)

Next, the W1/O/W2 emulsion was transferred into a lidless open glass container and stirred by a stirrer at room temperature for about 20 hours to evaporate hexane. After the solvent removal, the encapsulation ratio of cytarabine was 50%.

(Concentration of Liposomes by Substitution of Outer Aqueous Phase (W2))

The resulting liposome solution was subjected to ultrafiltration, and while removing the outer aqueous phase (W2), the same Tris-HCl buffer solution (w3) (pH: 8, 50 mmol/L) as the aqueous solvent (w1) was added to exclude cytarabine contained in the outer aqueous phase (W2). Finally, a liposome-containing preparation in an amount of 10 mL that was twice the volume (5 mL) of the inner aqueous phase liquid (W1) was produced. In this preparation, liposomes encapsulating cytarabine in an amount of 50% (20[mg/mL]×5[mL]×0.50=50 [mg]) of the feed amount were contained. The drug concentration was 5.0 mg/mL, and cytarabine was encapsulated in a proportion of 100% in liposomes. The drug weight ratio (d/f) was (50 [mg]/(300+152+108) [mg]=50/460=0.109.

### Reference Example B-1

A liposome-containing preparation was produced in the same manner as in Reference Example A-1, except that, of the stirring conditions, the number of revolutions per minute (n) was changed to 100 [rpm] (therefore, r×n/L′=0.03×100/50=0.06). After the solvent removal, the encapsulation ratio of cytarabine was 55%. That is to say, in the preparation after the ultrafiltration, liposomes encapsulating cytarabine in an amount of 55% (55 mg) of the feed amount were contained. The drug concentration was 5.5 mg/mL, and cytarabine was encapsulated in a proportion of 100% in liposomes. The drug weight ratio (d/f) was 55/460=0.120.

### Reference Example B-2

A liposome-containing preparation was produced in the same manner as in Reference Example A-1, except that the solubilizing aid was changed to trometamol from mannitol (the volume-average particle diameter of the resulting W1/O emulsion was 50 nm and the same), and of the stirring conditions, the radius (r) of the stirrer was changed to 0.003 [m] and the number of revolutions per minute (n) was changed to 1000 [rpm] (therefore, r×n/L′=0.003×1000/50=0.06). After the solvent removal, the encapsulation ratio of cytarabine was 51%. That is to say, in the preparation after the ultrafiltration, liposomes encapsulating cytarabine in an amount of 51% (51 mg) of the feed amount were contained. The drug concentration was 5.1 mg/mL, and cytarabine was encapsulated in a proportion of 100% in liposomes. The drug weight ratio (d/f) was 51/460=0.111.

### Reference Example B-3

A liposome-containing preparation was produced in the same manner as in Reference Example A-1, except that, of the stirring conditions, the radius (r) of the stirrer was changed to 0.0007 [m] and the number of revolutions per minute (n) was changed to 10000 [rpm] (therefore, r×n/L′=0.0007×10000/50=0.14). After the solvent removal, the encapsulation ratio of cytarabine was 49%. That is to say, in the preparation after the ultrafiltration, liposomes encapsulating cytarabine in an amount of 49% (49 mg) of the feed amount were contained. The drug concentration was 4.9 mg/mL, and cytarabine was encapsulated in a proportion of 100% in liposomes. The drug weight ratio (d/f) was 49/460=0.107.

### Reference Example A-2

A liposome-containing preparation was produced in the same manner as in Reference Example A-1, except that, of the stirring conditions, the radius (r) of the stirrer was changed to 0.0007 [m] and the number of revolutions per minute (n) was changed to 20000 [rpm] (therefore, r×n/L′=0.0007×20000/50=0.28). After the solvent removal, the encapsulation ratio of cytarabine was 40%. That is to say, in the preparation after the ultrafiltration, liposomes encapsulating cytarabine in an amount of 40% (40 mg) of the feed amount were contained. The drug concentration was 4.0 mg/mL, and cytarabine was encapsulated in a proportion of 100% in liposomes. The drug weight ratio (d/f) was 40/460=0.087.

### Reference Example A-3

A liposome-containing preparation was produced in the same manner as in Reference Example A-1, except that the solubilizing aid was changed to meglumine from mannitol, the “pulse ultrasonic irradiation” in the primary emulsification step was returned to “15 minute-ultrasonic irradiation” similarly to Example 1-1 (the volume-average particle diameter of the resulting W1/O emulsion was 190 nm), and of the stirring conditions in the secondary emulsification, the radius (r) of the stirrer was changed to 0.16 [m] and the number of revolutions per minute (n) was changed to 50 [rpm] (therefore, r×n/L′=0.16×50/190=0.04). After the solvent removal, the encapsulation ratio of cytarabine was 50%. That is to say, in the preparation after the ultrafiltration, liposomes encapsulating cytarabine in an amount of 50% (50 mg) of the feed amount were contained. The drug concentration was 5.0 mg/mL, and cytarabine was encapsulated in a proportion of 100% in liposomes. The drug weight ratio (d/f) was 50/460=0.107.

### Reference Example B-4

A liposome-containing preparation was produced in the same manner as in Reference Example A-3, except that, of the stirring conditions, the number of revolutions per minute (n) was changed to 100 [rpm] (therefore, r×n/L′=0.16×100/190=0.08). After the solvent removal, the encapsulation ratio of cytarabine was 55%. That is to say, in the preparation after the ultrafiltration, liposomes encapsulating cytarabine in an amount of 55% (55 mg) of the feed amount were contained. The drug concentration was 5.5 mg/mL, and cytarabine was encapsulated in a proportion of 100% in liposomes. The drug weight ratio (d/f) was 55/460=0.120.

### Reference Example B-5

A liposome-containing preparation was produced in the same manner as in Reference Example A-3, except that the solubilizing aid was changed to mannitol from meglumine (the volume-average particle diameter of the resulting W1/O emulsion was 190 nm and the same), and of the stirring conditions, the radius (r) of the stirrer was changed to 0.016 [m] and the number of revolutions per minute (n) was changed to 1000 [rpm] (therefore, r×n/L′=0.016×1000/190=0.08). After the solvent removal, the encapsulation ratio of cytarabine was 52%. That is to say, in the preparation after the ultrafiltration, liposomes encapsulating cytarabine in an amount of 52% (52 mg) of the feed amount were contained. The drug concentration was 5.2 mg/mL, and cytarabine was encapsulated in a proportion of 100% in liposomes. The drug weight ratio (d/f) was 52/460=0.113.

### Reference Example B-6

A liposome-containing preparation was produced in the same manner as in Reference Example A-3, except that the solubilizing aid was changed to trometamol from meglumine (the volume-average particle diameter of the resulting W1/O emulsion was 190 nm and the same), and of the stirring conditions, the radius (r) of the stirrer was changed to 0.0016 [m] and the number of revolutions per minute (n) was changed to 10000 [rpm] (therefore, r×n/L′=0.0016×10000/190=0.08). After the solvent removal, the encapsulation ratio of cytarabine was 42%. That is to say, in the preparation after the ultrafiltration, liposomes encapsulating cytarabine in an amount of 42% (42 mg) of the feed amount were contained. The drug concentration was 4.2 mg/mL, and cytarabine was encapsulated in a proportion of 100% in liposomes. The drug weight ratio (d/f) was 42/460=0.091.

### Reference Example A-4

A liposome-containing preparation was produced in the same manner as in Reference Example A-3, except that the solubilizing aid was changed to trometamol from meglumine (the volume-average particle diameter of the resulting W1/O emulsion was 190 nm and the same), and of the stirring conditions, the radius (r) of the stirrer was changed to 0.0016 [m] and the number of revolutions per minute (n) was changed to 20000 [rpm] (therefore, r×n/L′=0.0016×20000/190=0.16). After the solvent removal, the encapsulation ratio of cytarabine was 42%. That is to say, in the preparation after the ultrafiltration, liposomes encapsulating cytarabine in an amount of 42% (42 mg) of the feed amount were contained. The drug concentration was 4.2 mg/mL, and cytarabine was encapsulated in a proportion of 100% in liposomes. The drug weight ratio (d/f) was 42/460=0.091.

TABLE 5
Results of Reference Examples A-1 to A-4 and B-1 to B-6 carried out
by properly changing production scale using production container of similar figure
(stirring emulsification method, encapsulation of cytarabine)

| | L′ (W/O | n (number | | | Encapsulation | Particle size | |
|---|---|---|---|---|---|---|---|
| | particle | of revolutions | r (radius | | ratio | distribution | |
| | diameter | per minute | of stirrer | r × | after solvent | after solvent | Solubilizing |
| | [nm]) | [rpm]) | [m]) | n/L′ | removal | removal | aid |
| Ref. | 50 | 25 | 0.03 | 0.015 | 50% | plural | mannitol |
| Ex. A-1 | | | | | | peaks | (10 mg/mL) |
| Ref. | 50 | 100 | 0.03 | 0.06 | 55% | normal | mannitol |
| Ex. B-1 | | | | | | distribution | (10 mg/mL) |
| Ref. | 50 | 1000 | 0.003 | 0.06 | 51% | normal | trometamol |
| Ex. B-2 | | | | | | distribution | (1 mg/mL) |
| Ref. | 50 | 10000 | 0.0007 | 0.14 | 49% | normal | mannitol |
| Ex. B-3 | | | | | | distribution | (10 mg/mL) |
| Ref. | 50 | 20000 | 0.0007 | 0.28 | 40% | plural | mannitol |
| Ex. A-2 | | | | | | peaks | (10 mg/mL) |
| Ref. | 190 | 25 | 0.16 | 0.02 | 50% | plural | meglumine |
| Ex. A-3 | | | | | | peaks | (25 mg/mL) |
| Ref. | 190 | 100 | 0.16 | 0.08 | 55% | normal | meglumine |
| Ex. B-4 | | | | | | distribution | (25 mg/mL) |
| Ref. | 190 | 1000 | 0.016 | 0.08 | 52% | normal | mannitol |
| Ex. B-5 | | | | | | distribution | (10 mg/mL) |
| Ref. | 190 | 10000 | 0.0016 | 0.08 | 42% | normal | trometamol |
| Ex. B-6 | | | | | | distribution | (1 mg/mL) |
| Ref. | 190 | 20000 | 0.0016 | 0.16 | 42% | plural | trometamol |
| Ex. A-4 | | | | | | peaks | (1 mg/mL) |

1. A liposome-containing preparation which is a preparation containing univesicular liposomes that encapsulate a highly water-soluble drug (d) having a water solubility of higher than 10 mg/mL and have a volume-average particle diameter of 50 to 200 nm, wherein the highly water-soluble drug (d) and a solubilizing aid (s) having log D of not more than −1 at pH 7.4 are dissolved in an inner aqueous phase (W1) of the univesicular liposome.

2. The liposome-containing preparation as claimed in claim 1, wherein the drug concentration of the highly water-soluble drug (d) in the liposome-containing preparation is not less than 5 mg/mL.

3. The liposome-containing preparation as claimed in claim 1, wherein the weight ratio (d/f) of the highly water-soluble drug (d) to a lipid component (f) constituting liposomes is not less than 0.05.

4. The liposome-containing preparation as claimed in claim 1, wherein the highly water-soluble drug (d) is dissolved in a supersaturation state in the inner aqueous phase (W1).

5. A production process for a preparation containing univesicular liposomes that encapsulate a highly water-soluble drug (d) having a water solubility of higher than 10 mg/mL and have a volume-average particle diameter of 50 to 200 nm, said production process comprising the following steps (1) to (4):
(1) a primary emulsification step comprising emulsifying an oil phase liquid (O), in which a lipid component (f1) is dissolved in an organic solvent (o) that is volatile under the solvent removal conditions of the following step (3), and an aqueous phase liquid (W1), in which the highly water-soluble drug (d) and a solubilizing aid (s) having log D of not more than −1 at pH 7.4 are dissolved in an aqueous solvent (w1), to produce a W1/O emulsion,
(2) a secondary emulsification step comprising emulsifying the W1/O emulsion obtained through the step (1) and an aqueous phase liquid (W2) to produce a W1/O/W2 emulsion,
(3) a solvent removal step comprising removing the organic solvent (o) contained in the oil phase liquid (O) from the W1/O/W2 emulsion obtained through the step (2) to form liposomes, and
(4) an aqueous phase substitution step comprising removing the aqueous phase liquid (W2) from a liposome dispersion obtained through the step (3) and adding an aqueous phase liquid (W3) to produce a liposome preparation.

6. The process as claimed in claim 5, wherein the secondary emulsification in the step (2) is carried out by a stirring emulsification method satisfying the condition of the following formula (e1):
0.02385<r×n/L′<0.1431 (e1)
wherein r represents a radius [m] of a stirrer, L′ represents a particle diameter [nm] of the W1/O emulsion, and n represents a number of revolutions per minute [rpm] of the stirrer.

7. The production process as claimed in claim 5, wherein the liposome dispersion is concentrated in the step (4) so that the drug concentration of the highly water-soluble drug (d) in the liposome-containing preparation might become not less than 5 mg/mL.

8. The production process as claimed in claim 5, wherein in the liposome-containing preparation obtained through the step (4), the weight ratio (d/f) of the highly water-soluble drug (d) to the lipid component (f) constituting liposomes is not less than 0.05.

9. The production process as claimed in claim 8, wherein the aqueous phase liquid (W1) in which the highly water-soluble drug (d) is dissolved in a supersaturation state in the aqueous solvent (w1) is used in the step (1).

10. The production process as claimed in claim 5, wherein the aqueous phase liquid (W2) in which a water-soluble emulsifying agent (r) is dissolved is used in the step (2).

11. The production process as claimed in claim 5, wherein all of the steps (1) to (4) are carried out at a temperature in the range of 5 to 10° C.

12. The production process as claimed in claim 5, wherein the primary emulsification in the step (1) is carried out using pulse ultrasonic waves.
